(19) United States
(12) Patent Application Publication
(10) Pub. No.: US 2025/0277804 A1
(43) Pub. Date: Sep. 4, 2025

(54) SYSTEM FOR AUTOMATED HIGH-SPEED IMAGING AND GENETIC MANIPULATION OF SMALL ANIMALS

(71) Applicant: Ohio State Innovation Foundation, Columbus, OH (US)

(72) Inventors: Christopher Fang-Yen, Columbus, OH (US); Serge Alhalbi, Columbus, OH (US); Zihao Li, Columbus, OH (US)

(21) Appl. No.: 19/069,454

(22) Filed: Mar. 4, 2025

Related U.S. Application Data
(60) Provisional application No. 63/561,057, filed on Mar. 4, 2024.

(51) Int. Cl. G01N 35/00; C12Q 1/04; C12Q 1/24; G01N 21/64; G01N 35/10; G06T 7/00

(52) U.S. Cl. CPC G01N 35/0099; C12Q 1/04; C12Q 1/24; G01N 21/6458; G01N 35/00069; G01N 35/00732; G01N 35/1004; G06T 7/0012; G01N 2035/00099; G06T 2207/10056

(57) ABSTRACT

A system includes an articulated robotic arm having a plate manipulator and an organism manipulator. A shelf array holds a plurality of plates within reach of the plate manipulator of the robotic arm. The shelf array includes plate shelves and lid shelves configured to retain plates and lids, respectively, including a first plate that holds a target organism. The system includes a working platform for organizing and supporting the plates on plate stations. The plate manipulator of the robotic arm is moveable to engage with the first plate to (i) remove the first plate from the first plate shelf, (ii) deposit the first lid from the first plate onto the first lid shelf, and (iii) move the first plate to a first plate station on the working platform without disengaging from the first plate. The organism manipulator can view and move organisms within the first plate.

### CROSS-REFERENCE TO RELATED APPLICATIONS

This application claims priority to U.S. Provisional Patent Application No. 63/561,057, filed Mar. 4, 2024, the entire contents of which are incorporated herein by reference.

### GOVERNMENT LICENSES RIGHTS

This invention was made with government support under R01 NS115995 awarded by the National Institutes of Health. The government has certain rights in the invention.

### BACKGROUND

The present disclosure relates generally to research procedures using small animals (e.g., <10 mm animals such as Caenorhabditis elegans (C. elegans), other nematodes such as Caenorhabditis briggsae, Drosophila larvae, or other organisms) and the systems utilized in their transport and analysis.

The free-living roundworm Caenorhabditis elegans (C. elegans) is a powerful model organism in biology, known for its advantages such as rapid development, compact anatomy, optical transparency, and genetic manipulability. However, many C. elegans procedures carried out in laboratories have traditionally relied on labor-intensive and time-consuming manual procedures. Manual procedures are labor intensive because they usually require animals to be manipulated individually. This becomes particularly challenging when conducting experiments involving a large number of animals and/or groups of animals. When planning experiments, the use of manual procedures imposes practical limits on the number of conditions and the number of replicates for each condition, thereby weakening the statistical power of the experiments. Finally, manual procedures require training and are prone to errors. This reliance on investigator-acquired skills can be a barrier for scientists without prior experience with C. elegans who wish to use this system.

Therefore, a need exists for improved systems for transporting and analyzing organisms.

### SUMMARY

According to one implementation, a system is disclosed, the system including an articulated robotic arm, a plurality of plats, a first shelf array, and a working platform. The articulated robotic arm (e.g., 6-axis robotic arm) includes at least one end effector including a plate manipulator. The plurality of plates (e.g., petri dishes or agar plates) includes a first plate having a first diameter. The first plate includes a target organism. A first lid corresponding to the first plate is removably coupled to the first plate. The first lid has a second diameter that is larger than the first diameter such that the first lid covers the first plate. The first shelf array includes a plurality of shelving units arranged within reach of the plate manipulator of the robotic arm. The first shelf array includes a first plate shelf configured to retain the first plate. The first plate shelf defines a first plate holder opening having a third diameter. The third diameter of the first plate holder opening is less than the first diameter of the first plate. The first shelf array further includes a first lid shelf configured to retain the first lid. The first lid shelf defines a first lid holder opening having a fourth diameter. The fourth diameter of the first lid holder opening is less than the second diameter of the first lid and greater than the first diameter of the first plate. The working platform for organizing and supporting the plurality of plates includes an upper surface defining a plurality of plate stations. The plate manipulator of the robotic arm is moveable to engage with the first plate to (i) remove the first plate from the first plate shelf, (ii) deposit the first lid from the first plate onto the first lid shelf, and (iii) move the first plate to a first plate station of the plurality of plate stations on the working platform without disengaging from the first plate.

In some implementations, the plurality of plates further includes a second plate with a corresponding second lid. The first shelf array further includes a second plate shelf configured to retain the second plate and a second lid shelf configured to retain the second lid. The plate manipulator of the robotic arm is moveable to engage with the second plate to (i) remove the second plate from the second plate shelf, (ii) deposit the second lid from the second plate onto the second lid shelf, and (iii) move the second plate to a second plate station of the plurality of plate stations on the working platform without disengaging from the first plate.

In some implementations, the plate manipulator includes a plate manipulator width that is less than the third diameter of the first plate holder opening such that the plate manipulator can pass through the first plate holder opening and the first lid holder opening without interference.

In some implementations, when removing the first plate from the first plate shelf, the plate manipulator moves upward through the first plate holder opening to engage with a base of the first plate.

In some implementations, when depositing the first lid onto the first lid shelf, the plate manipulator moves downward through the first lid holder opening such that the first lid shelf engages with and removes the first lid while the first plate passes through the first lid holder opening with the plate manipulator.

In some implementations, the plurality of plate stations includes corresponding openings defined in the upper surface of the working platform, wherein a diameter of the openings of the plurality of plate stations are smaller than the first diameter of the first plate and larger than a plate manipulator width of the plate manipulator.

In some implementations, when moving the first plate to the first plate station, the plate manipulator moves through a first opening defined by the first plate station to deposit the first plate onto the first plate station.

In some implementations, the at least one end effector further includes a wire-loop picking tool including a wire loop electrically coupled to a power source to sterilize the wire-loop between uses, wherein each of the plate manipulator and the wire-loop picking tool is moveable between an active position and an inactive position relative to the robotic arm.

In some implementations, the at least one end effector further includes a camera adjacent to the wire-loop picking tool.

In some implementations, the camera is configured to identify the target organism from the first plate and the wire-loop picking tool is configured to retrieve the target organism from the first plate based on information from the camera.

In some implementations, the system further includes a controller in communication with the robotic arm, wherein the controller includes a processor and a memory storing instructions thereon that, when executed by the processor, cause the robotic arm to perform a plate manipulation operation of an organism manipulation operation.

In some implementations, the memory further stores a location for each of the plurality of plates held within the array of shelves.

In some implementations, the system further includes an imaging system located under the working platform, the imaging system including at least one camera moveable along one or more rails, wherein the at least one camera is alignable with any one of the plurality of plate stations and a corresponding opening thereof to capture images of a plate therein.

In some implementations, the imaging system further includes a microscope coupled with a fluorescent imaging apparatus.

In some implementations, the system further includes a controller in communication with the robotic arm and the imaging system, the controller including a processor and a memory storing instructions thereon that, when executed by the processor, cause the robotic arm to perform a plate manipulation operation of an organism manipulation operation, wherein images captured by the imaging system are delivered to the controller and analyzed to identify the target organism or attributes thereof.

According to another implementation, a system disclosed, the system including an articulated robotic arm, a first shelf array, a working platform, and an imaging system. The articulated robotic arm (e.g., 6-axis robotic arm) includes at least one end effector. The first shelf array includes a plurality of shelving units arranged within reach of the at least one end effector of the robotic arm. The first shelf array houses a plurality of plates (e.g., petri dishes or agar plates). The at least one end effector is moveable to remove a first plate of the plurality of plates from the first shelf array. The working platform for organizing and supporting the plurality of plates includes an upper surface defining a plurality of plate stations each configured to retain one of the plurality of plates. The plurality of plate stations includes corresponding openings defined in the upper surface of the working platform. The working platform further includes a lower cavity defined underneath the working surface, the lower cavity being in fluid communication with each of the openings of the plurality of plate stations. The imaging system is located within the lower cavity. The imaging system includes at least one rail arranged across the lower cavity, a carriage movably coupled to the at least one rail, and an optical detection device movably coupled to the carriage. The optical detection device includes an objective lens alignable with each of the plurality of plate stations.

In some implementations, the robotic arm is configured to deposit the first plate onto a first plate station of the plurality of plate stations, wherein the objective lens of the imaging system is selectively alignable with a lower surface of the first plate to capture images of the first plate and a target organism therein.

In some implementations, the system further includes a controller in communication with the robotic arm and the imaging system, wherein the controller is configured to receive images of the first plate and the target organism from the imaging system and analyze the images to identify and characterize the target organism.

In some implementations, the controller is configured to identify features of the target organism.

In some implementations, the controller is configured to identify, via machine vision algorithms and artificial intelligence, one or more phenotypes of the target organism.

In some implementations, the at least one end effector includes a plate manipulator configured to engage with and move the first plate.

In some implementations, the at least one end effector includes a wire-loop picking tool.

In some implementations, the at least one end effector further includes a camera adjacent to the wire-loop picking tool.

According to another implementation, a method of categorizing a target organism is disclosed. The method includes (i) providing a system including: a plurality of plates including a first plate containing a target organism and a first lid removably coupled to the first plate; a first shelf array configured to house the plurality of plates, the first shelf array including a first plate shelf and a first lid shelf; a working platform including a plurality of plate stations arranged along an upper surface and configured to hold the plurality of plates, the working platform defining a lower cavity accessible at least via an opening corresponding to each of the plurality of plate stations; an imaging system movable along one or more rails disposed within the lower cavity, the imaging system including at least one optical device; a robotic arm including at least one end effector moveable between the array of shelves and the working platform; and a controller in communication with the robotic arm and the imaging system. The method further includes (ii) retrieving, via the at least one end effector of the robotic arm, the first plate from the first shelf. The method further includes (iii) depositing, via the at least one end effector of the robotic arm, the first plate onto a first plate station of the plurality of plate stations of the working platform. The method further includes (iv) actuating, via the controller, the imaging system to align the at least one optical device with the first plate. The method further includes (v) capturing, via the at least one optical device, images of the target organisms in the first plate. The method further includes (vi) identifying, via the controller, an attribute of the target organism.

In some implementations, depositing the first plate onto the first plate station includes: moving the at least one end effector to the first plate shelf housing the first plate, wherein the at least one end effector is a plate manipulator; lifting, via the plate manipulator, the first plate and the first lid off of the first plate shelf; moving the first plate through an opening of the first lid shelf to deposit the first lid on the first lid shelf, exposing the contents of the first plate; and moving the first plate to the first plate station.

In some implementations, identifying an attribute of the target organism includes: analyzing, via machine vision and/or artificial intelligence systems, images of the target organisms; and identifying one or more phenotypes of the target organism.

In some implementations the at least one end effector further includes a wire-loop picking tool, the method further including: moving, via the robotic arm, the wire-loop picking tool into proximity with the first plate; identifying, via the at least one optical device of the imaging system or a camera coupled to the wire-loop picking tool, a first target organism; contacting the first target organism with the wire-loop picking tool; and moving and depositing, via the robotic arm, the first target organism into a second plate on a second plate station.

This summary is illustrative only and is not intended to be in any way limiting. Other aspects, features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The systems, methods, and devices are explained in even greater detail in the following drawings. The drawings are merely exemplary and certain features may be used singularly or in combination with other features. The drawings are not necessarily drawn to scale.

FIG. 1 is an isometric view of a plate manipulation system, according to one implementation.

FIG. 2 is a top view of the plate manipulation system of FIG. 1.

FIG. 3 is a detail view of the end effectors of the robotic arm, according to one implementation.

FIG. 4 is a detail view of the plate storage system (e.g., “plate hotel”), including three shelves for plates (below) and three shelves for plate lids (above), according to one implementation.

FIG. 5A is a diagram showing an operation to remove a plate from the plate storage shelf using a plate manipulator, according to one implementation.

FIG. 5B is a diagram showing a lid removal operation, according to one implementation.

FIG. 6 is an isometric view of a working platform of the plate manipulation system, and a microscope under the platform, according to one implementation.

FIG. 7 is an isometric view of a rail system supporting a motorized carriage with a microscope optical system device, according to one implementation.

FIG. 8 is a side view of the optical system of FIG. 7, according to one implementation.

FIG. 9 shows a dark field image and a bright field image taken from a camera mounted to the robotic arm and shows a wire loop in a field of living organisms, according to an exemplary implementation.

FIGS. 10A-10F show images taken from a camera mounted to the robotic arm showing an organism manipulation operation wherein a wire loop contacts and transfers a C. elegans worm, according to an exemplary implementation.

FIG. 11 shows a high-resolution image of several C. elegans taken from a microscope underneath the work station, according to an exemplary implementation.

FIG. 12A shows an exemplary operation of a worm-picking system and optical system thereof, according to one implementation.

FIG. 12B shows an exemplary worm-picking system, according to one implementation.

FIG. 12C shows a wire loop being sterilized by an electric current, according to one implementation.

FIGS. 12D and 12E show a schematic of pick motion trajectories enabling C. elegans to be picked up from and put down to an agar substrate by the wire loop, according to one implementation.

FIG. 12F shows a timeline for stages throughout an automated transfer along with high-magnification frames of key points, according to one implementation.

FIG. 13A shows a machine vision image showing the analysis and tracking of worms in real time for low-magnification frames, according to one implementation.

FIG. 13B shows a machine vision image of a high-magnification frame showing bright field phenotyping, according to one implementation.

FIG. 13C shows a machine vision image of a high-magnification frame showing fluorescence phenotyping, according to one implementation.

FIGS. 14A-14C show green fluorescent protein (GFP) images of a GFP-expressing transgenic strain, a dumpy mutant, and a cross between the GFP-expressing strain and the dumpy mutant C. elegans, according to various implementations.

FIG. 14D shows a schematic of how an automated genetic cross procedure was implemented, according to one implementation.

FIG. 15A shows an image of automated genetic mapping of a red fluorescent protein (RFP) transgene in C. elegans for an RFP expression in the strain carrying the transgene of interest, according to one implementation.

FIG. 15B shows an image of automated genetic mapping for a GFP expression in the genetic balancer strain, according to another implementation.

FIG. 15C shows a schematic of a linkage test, according to various implementations.

FIG. 15D shows a linkage index graph for various chromosomes and genetic balancers, according to one implementation.

FIG. 16 shows a diagram of the procedures for an automated genomic integration of a green fluorescence-labeled extrachromosomal array, according to one implementation.

### DETAILED DESCRIPTION

Following below are more detailed descriptions of concepts related to, and implementations of, methods, apparatuses, and systems for the transport, organization, and/or analysis of organisms (e.g. C. elegans disposed on a petri dish). The figures illustrate exemplary implementations in detail and the present disclosure is not limited to the details or methodology set forth in the description or illustrated in the figures. The terminology used herein is for the purpose of description only and should not be regarded as limiting.

### Example System

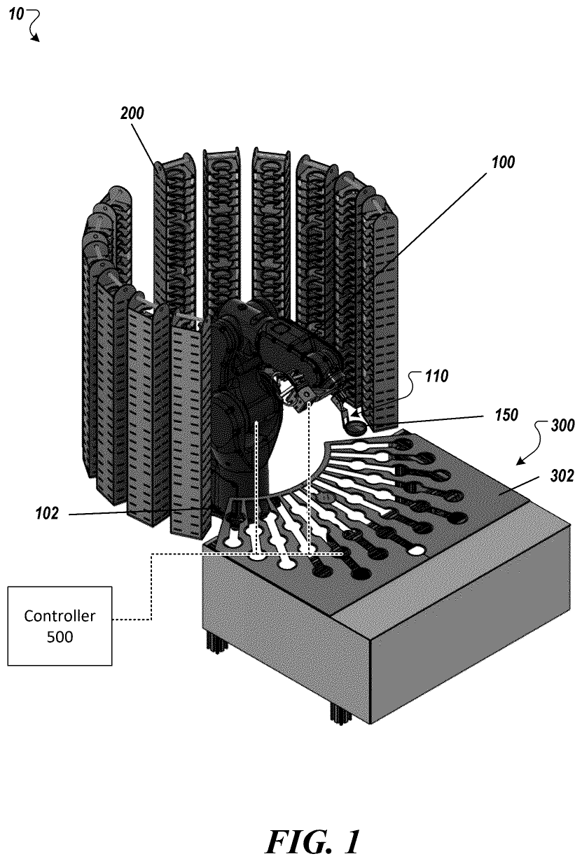

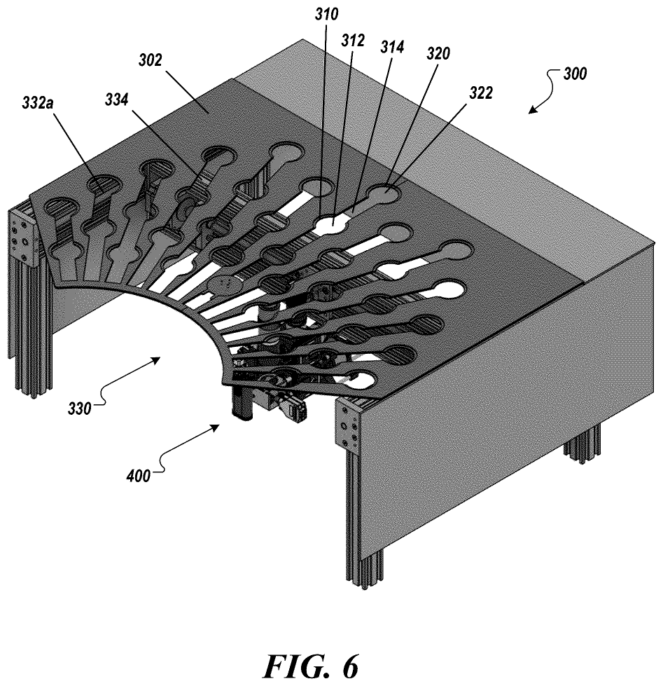

FIG. 1 shows a system 10 for transporting and analyzing target organisms, according to an exemplary implementation. The system 10 includes an articulated robotic arm 100, a plurality of shelf arrays including a first shelf array 200 supporting a plurality of plates, a working platform 300, an imaging system 400 (FIG. 6), and a controller 500.

The robotic arm 100 is arranged between the working platform 300 and the plurality of shelf arrays. The robotic arm 100 is an articulable and controllable robotic arm having several members connected to each other at rotatable joints. The robotic arm 100 includes a base 102 secured to the floor or other base surface of the system 10. The robotic arm 100 is rotatable about the base 102.

The articulated robotic arm 100 includes an end effector 104 disposed on an end of the robotic arm 100 opposite from the base 102. The robotic arm 100 is movable to reach each of the plurality of shelf arrays and at least a majority of the working platform 300. Specifically, the various members and portions of the robotic arm 100 are moveable to position the end effector 104 at a desired position in the system 10.

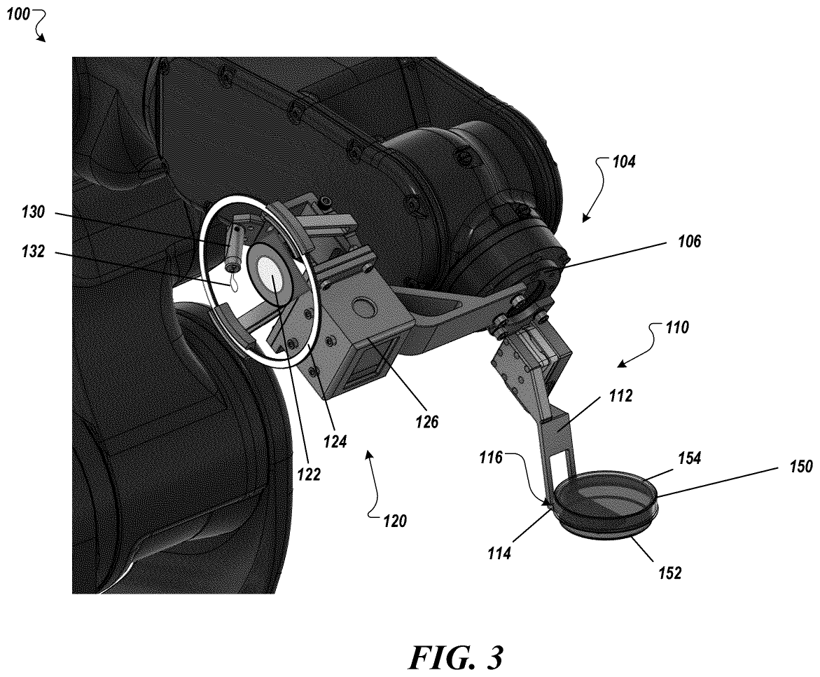

As shown in FIG. 3, the end effector 104 may include one or more different types of end effectors coupled to a rotatable end effector plate 106 on the end of the robotic arm 100. For example, the end effector plate 106 is rotatable to move one of the end effectors into an active position and another of the end effectors into an inactive position. FIG. 3 shows two end effectors coupled to the end effector plate 106. However, in other implementations, the robotic arm may include one or more end effectors removably coupled thereto. Each of the end effectors described herein may be coupled to the end effector plate 106 via a magnetic mount, fasteners, or other connection methods.

A plate manipulator 110 is coupled to the end effector plate 106 and shown in the active position in FIG. 3. The plate manipulator 110 includes a connector portion 112 that extends distally from the end effector plate 106 of the robotic arm 100. The plate manipulator 110 includes a lift portion 114 extending distally from the connector portion 112 in a direction transverse to that of the connector portion 112. The connector portion 112 and the lift portion 114 form an angle between each other such that the plate manipulator 110 approximates a spatula. The lift portion 114 includes a lift surface 116 that may be oriented substantially horizontally (e.g., parallel to, or substantially parallel to a floor surface) during normal operation of the robotic arm 100 (e.g., when transporting a plate). However, the lift surface 116 and other portions of the plate manipulator 110 may be rotated, angled, or moved in a variety of positions and orientations (e.g., within 6 degrees of freedom relative to the base 102 of the robot arm 100). The lift portion 114 includes a width (e.g., a plate manipulator width). The width of the lift portion 114 and/or other portions of the plate manipulator 110 may be configured based on a dimension of the first shelf array 200 and/or a plate therein.

The plate manipulator 110 and the lift portion 114 thereof are configured to support and transport a plate (e.g., a petri dish or an agar plate). For example, a first plate 150 is shown in FIGS. 1-3 being supported by the lift portion 114 of the plate manipulator 110. The first plate includes a first base 152 defining a well or cavity within which one or more target organisms may be retained. For example, agar containing C. elegans may be retained within the well of the first base 152. The lift surface 116 of the lift portion 114 engages with first base 152 of the first plate 150. The first plate 150 further includes a first lid 154 removably coupled to the first base 152. The first lid 154 extends across the first base 152 to cover the well containing the target organism and associated material. The first lid 154 includes a lid diameter that is larger than a base diameter of the first base 152 such that the first lid 154 extends over the edges of the first base 152. The first plate 150 and similar plates of the system 10 are further described herein with reference to other elements of the system 10. In some implementations, a camera and lens may be coupled to and/or positioned adjacent to the plate manipulator. For example, a camera and lens may provide an image of the plate in order to read a printed bar code label to aid with identification and indexing of the plate.

An organism manipulator 120 is coupled to the end effector plate 106 and shown in the inactive position in FIG. 3. The organism manipulator 120 includes several devices operating with each other to view and manipulate a target organism (e.g., C. elegans within a petri dish). For example, the organism manipulator 120 includes a wire-loop picking tool 130. The wire loop 132 is formed at a distal end of the wire-loop picking tool 130. The wire-loop picking tool 130 includes a wire loop 132 comprising an electrically conductive material such as a platinum-iridium alloy. The wire loop 132 is coupled to and in electrical communication with an electrical power source (e.g., a battery or other power source local to the organism manipulator 120). The power source is configured to provide electrical energy to the wire loop 132 to heat and sterilize the wire loop 132 (e.g., between uses or manipulation operations).

The organism manipulator 120 further includes a camera 122 adjacent to the wire-loop picking tool 130. The camera 122 is oriented and directed toward the wire loop 132. The camera 122 is configured to capture images (e.g., high-resolution images) of the wire loop 132, agar plate, and organisms contained therein. The organism manipulator 120 further includes an illumination device 124 positioned adjacent to the camera 122 and the wire loop 132. For example, the illumination device 124 of FIG. 3 is a ring light (e.g., LED ring light) positioned to illuminate the wire loop 132 and its surroundings.

The organism manipulator 120 further includes an electronics box 126. The electronics box 126 is coupled to and in electrical communication with each of the wire-loop picking tool 130, the camera 122, and the illumination device 124. For example, the electronics box 126 includes connections to a power source to supply power to each of the wire-loop picking tool 130, the camera 122, and the illumination device 124. Additionally, the electronics box 126 may include a wire or other data connection to the camera 122 to collect data (e.g., image data) from the camera 122. The electronics box 126 may further include a controller configured to operate each of the wire-loop picking tool 130, the camera 122, and/or the illumination device 124. In some implementations, the electronics box may include a controller in wireless communication with a remote controller (e.g., controller 500) configured to send and receive signals and/or data from the organism manipulator 120 and the elements thereof.

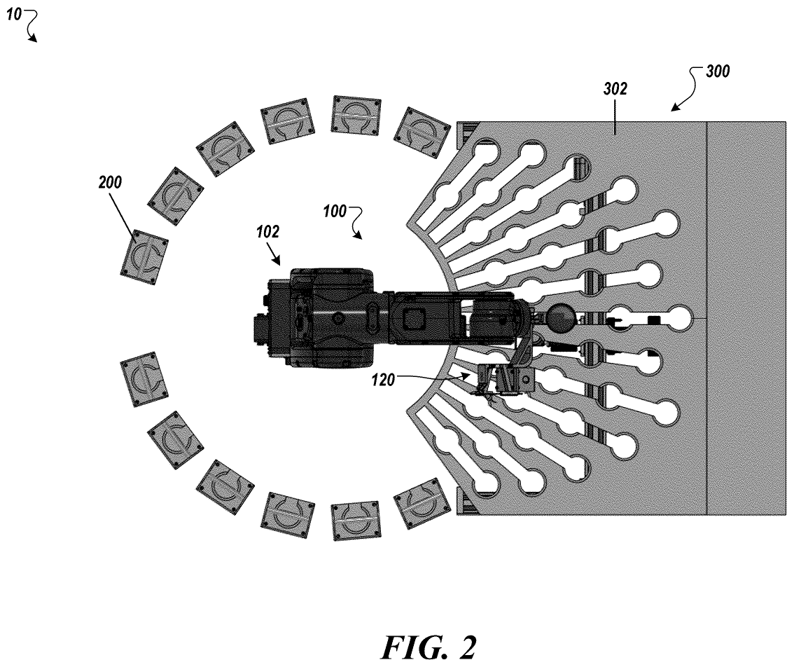

As shown in FIGS. 1 and 2, a plurality of shelf arrays encircles the robotic arm 100, including a first shelf array 200. The first shelf array 200 is substantially similar to each of the other shelf arrays shown; however, in other implementations, the other shelf arrays may have a different size, geometry, and/or orientation. Reference is made to the first shelf array 200 and elements thereof with the understanding that the same may be applied to any of the other shelf arrays.

The first shelf array 200 is configured to house and/or support a plurality of plates (e.g., petri dishes or agar plates). For example, the first shelf array 200 is configured to house the first plate 150 shown in FIGS. 1-3 and a plurality of substantially similar plates. In other implementations, the plurality of plates housed in the first shelf array may have different geometry, functions, and contents from each other.

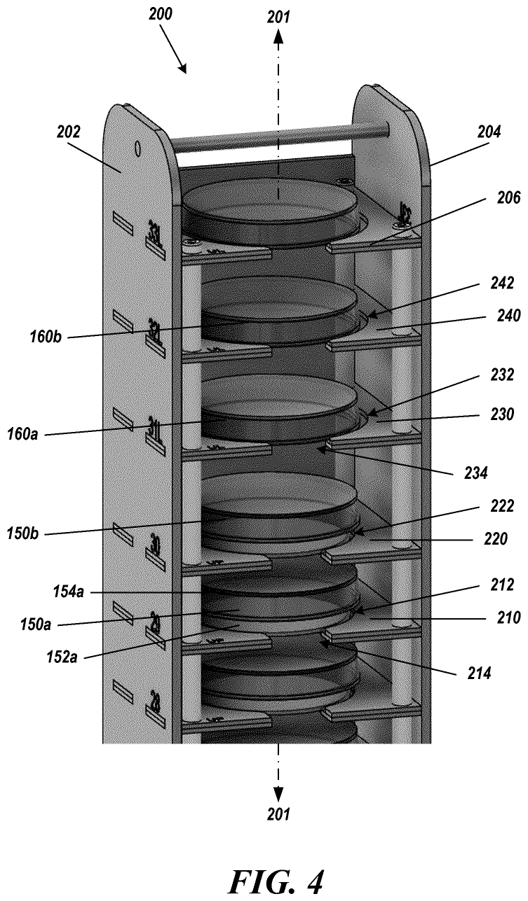

The first shelf array 200 includes first side wall 202 and a second side wall 204 spaced apart from the first side wall 202. A front side 206 of the first shelf array 200 is left open (i.e., without sidewalls). A plurality of shelving units extend between the first and second walls 202, 204. The plurality of shelving units arranged vertically to one another along a shelf axis 201, as shown in FIG. 4. The shelving units of the first shelf array 200 may be plate shelves or lid shelves. For example, as shown in FIG. 4, a first plate shelf 210 is arranged below a second plate shelf 220. A first lid shelf 230 is arranged above the second plate shelf 220. A second lid shelf 240 is arranged above the first lid shelf 230. The first shelf array 200 includes a plurality of lid shelves and plate shelves, but only a portion thereof is described herein. In other implementations, the order and sequencing of the plate and lid shelves may be different (e.g., alternating).

The first plate shelf 210 is configured to retain a plate (e.g., the first plate 150). As shown in FIG. 4, the first plate shelf 210 retains a plate 150a, and the second plate shelf 220 retains a plate 150b, each being substantially similar to the first plate 150 of FIGS. 1-3. The first plate shelf 210 defines a first plate holder opening 212. The first plate holder opening 212 has a substantially circular shape with a passthrough channel 214 on the front side 206 of the first plate shelf 210. The first plate holder opening 212 has a diameter that is less than the diameter of the base 152a of the plate 150a. Thus, base 152a of the plate 150a cannot pass through the first plate holder opening 212. Additionally, the passthrough channel 214 has a width that is greater than the width of the lift portion 114 of the robotic arm 100. Thus, the lift portion 114 of the robotic arm 100 can pass through the first plate holder opening 212 of the first plate shelf 210 without interference. The second plate shelf 220 defines a similar, second plate holder opening 222.

The first lid shelf 230 is configured to retain a lid of a plate (e.g., the first lid 154 of the first plate 150). As shown in FIG. 4, the first lid shelf 230 retains a lid 160a, and the second lid shelf 240 retains a lid 160b, each being substantially similar to the first lid 154 of the first plate 150 of FIGS. 1-3. The first lid shelf 230 defines a first lid holder opening 232. The first lid holder opening 232 has a substantially circular shape with a passthrough channel 234 on the front side 206 of the first lid shelf 230. The first lid holder opening 232 has a diameter that is less than the diameter of the lid 160a. Thus, the lid 160a cannot pass through the first lid holder opening 232. The diameter of the first lid holder opening 232 is also larger than that of the base 152a such that the base 152a of the plate 150a can pass through the first lid holder opening 232. Additionally, the passthrough channel 234 has a width that is greater than the width of the lift portion 114 of the robotic arm 100. Thus, the lift portion 114 of the robotic arm 100 can pass through the first lid holder opening 232 of the first lid shelf 230 without interference.

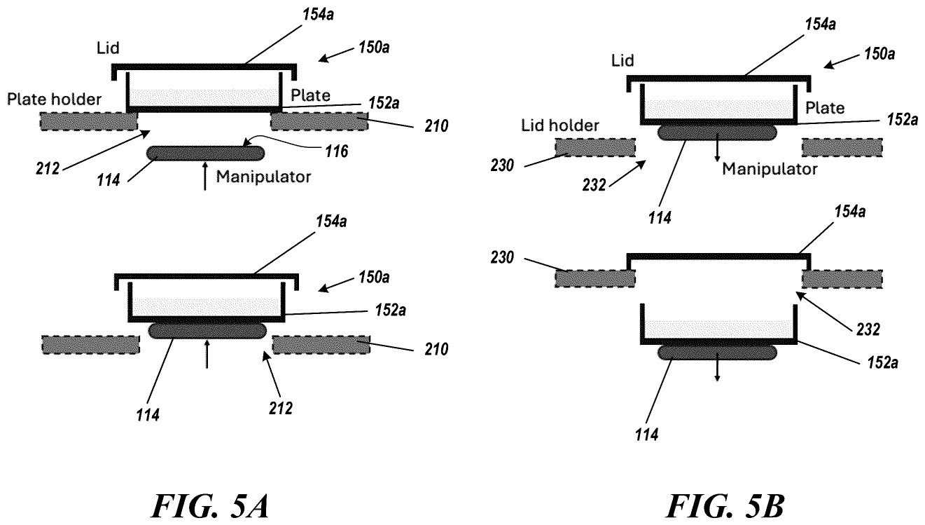

The geometry and arrangement of the first shelf array 200 and the plates therein may be better understood with reference to FIGS. 5A and 5B. The diagrams of FIGS. 5A and 5B show a side view of the first plate shelf 210 and the first lid shelf 230 with the plate 150a having the base 152a and a corresponding lid 154a. As shown in FIG. 5A in the top panel, the plate 150a sits on the first plate shelf 210. The diameter (or similar dimension) of the first plate holder opening 212 is greater than that of the base 152a. Thus, the base 152a and the lid 154a thereon are supported by the first plate shelf 210. In the bottom panel of FIG. 5A, the lift portion 114 of the plate manipulator 110 is inserted underneath the first plate shelf 210 and moved upward until the lift surface 116 engages with the base 152a. The lift portion 114 passes through the first plate holder opening 212 and lifts the plate 150a—including the base 152a and the corresponding lid 154a—off of the first plate shelf 210.

In FIG. 5B, the lift portion 114 of the plate manipulator 110 is moved, via the robotic arm 100, to the first lid shelf 230. The lift portion 114 carrying the plate 150a is moved downwards through the first lid holder opening 232 of the first lid shelf 230. Because the diameter (or similar dimension) of the first lid holder opening 232 is larger than that of the base 152a and the lift portion 114, the base 152a and the lift portion 114 pass through the first lid holder opening 232. However, the diameter (or similar dimension) of the first lid holder opening 232 is less than that of the corresponding lid 154a. Thus, the lid 154a is deposited on the first lid shelf 230. The plate base 152a containing the agar and/or target organisms is then ready to be moved to the work station 300 for manipulation of the animals therein.

The process shown in FIGS. 5A-5B may be performed in reverse. For example, the lift portion 114 may be moved upwards through the first lid holder opening 232 (e.g., from the bottom panel of FIG. 5B to the top panel of FIG. 5B) to re-couple the lid 154a onto the base 152a. Then, the lift portion 114 may be moved downwards through the first plate holder opening 212 to deposit the covered plate 150a onto the first plate shelf 210.

The process shown in FIGS. 5A-5B is a plate manipulation operation wherein the robotic arm 100 is used to pick up a plate 150a and remove the lid 154a of the plate 150a. The entire process of moving the plate manipulator 110 towards the first shelf array 200, picking a specific plate, removing its lid, and moving the lidless plate to the working platform may be entirely automated by the system 10. Thus, the system 10 does not involve the manual labor involved with individually selecting, removing the lids, using, returning the lid, and replacing a plate. The geometries of the plates and the shelves of the first shelf array 200 may be adjusted depending on the desired operation. Additionally, the locations of all plates in the system 10 may be tracked to facilitate automatic retrieval and replacement, for example, with the aid of bar codes attached to the plates for machine vision-based identification of plates.

The working platform 300 shown in FIG. 6 is configured to organize and support the plurality of plates (e.g., the first plate 150 or the plate 150a of FIGS. 4-5B). For example, in a plate manipulation operation of FIGS. 5A-5B, the lift portion 114 may move an exposed base 152a to the working platform 300 for further manipulation and/or study.

The working platform 300 includes an upper surface 302. The upper surface 302 defines a plurality of plate stations. The plurality of plate stations are arranged in rows extending radially from the robotic arm 100 so that the plate manipulator 110 and/or the organism manipulator 120 can reach each of the plate stations.

The plurality of plate stations includes a first plate station 310 and a second plate station 320. The first plate station 310 defines a first plate station opening 312 that is substantially circular. The diameter of the first plate station opening 312 is less than that of any one of the plurality of plates (e.g., the first plate 150). Thus, the first base 152 of the first plate 150 may be disposed on top of the first plate station 310. The second plate station 320 defines a second plate station opening 322 that is substantially similar to the first plate station opening 312. The second plate station opening 322 is formed with and/or in fluid communication with the first plate station opening 312 via a channel 314 defined to extend between the first plate station opening 312 and the second plate station opening 322. The channel 314 is provided at least to accommodate movement of the lift portion 114 when depositing or removing a plate from one of the plate stations.

The working platform 300 further includes a cavity 330 defined in part by the upper surface 302. The cavity 330 is positioned below the upper surface 302 and the plate stations thereof. The cavity 330 is in fluid communication with the first plate station opening 312 and the second plate station opening 322. For example, when the base 152a is positioned on the first plate station 310, the base 152a is viewable from within the cavity 330 through the first plate station opening 312.

The cavity 330 contains a first rail 332a and a second rail 332b extending in a first direction. The cavity 330 further contains a third rail 334 extending between the first and second rails 332a, 332b in a second direction perpendicular to the first direction. The rails are configured as a linear positioning system (e.g., an x-y linear positioning stage). The third rail 334 moves along the first and second rails 332a, 332b in the first direction. Furthermore, a carriage 340 mounted to the third rail 334 moves along the third rail 334 in the second direction. Thus, both the carriage 340 and the third rail 334 are moveable via actuators (e.g., electric motors) controllably coupled to one of the rails or a lead screw thereof. Thus, the position of the carriage 340 is controllable to move to any position in the cavity 330 underneath the plurality of plate stations (e.g., via controller 500).

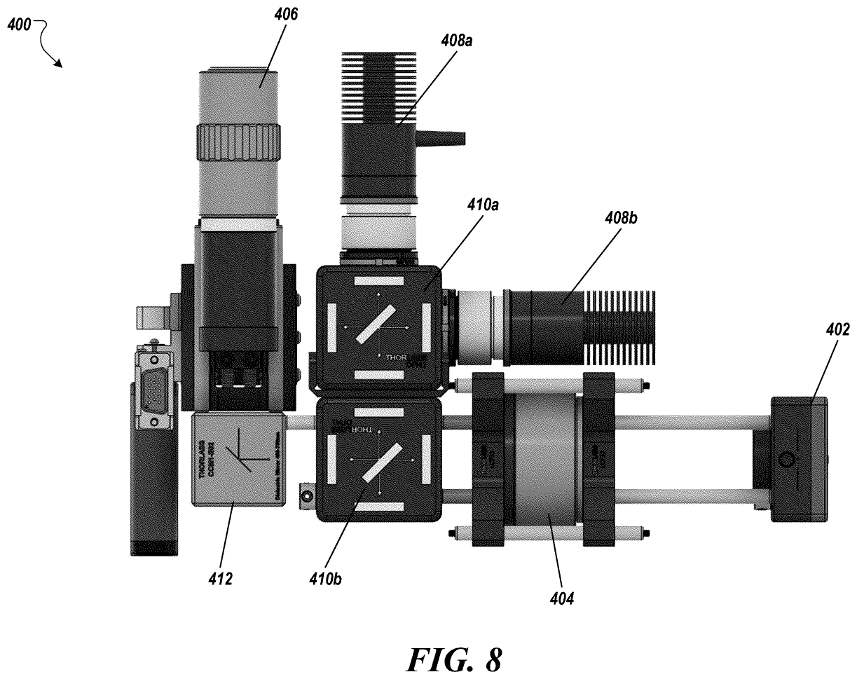

The carriage 340 supports the imaging system 400 (e.g., microscope system). The imaging system 400 includes one or more optical detection devices alignable with any one of the plate station openings (e.g., the first plate station opening 312 of the first plate station 310) of the working platform 300. As shown in FIG. 8, the imaging system 400 includes a camera 402. The camera 402 is configured to collect image data (e.g., high-resolution images) from the plates disposed on the working platform 300. The camera 402 may be further configured to output or send image data to a remote controller of the system (e.g., via wired or wireless connection from a controller thereof to controller 500).

The imaging system 400 further includes a tube lens 404 and an objective lens 406. The tube lens 404 and the objective lens 406 work in conjunction to view a portion of the plate (e.g., the first plate 150) and the contents thereof. For example, the tube lens 404 may have a focal length (f) of 100 mm, and the objective lens 406 may have a focal length (f) of 20 mm. The imaging system 400 further includes a mirror cube 412 aligned with the camera 402, the tube lens 404, and the objective lens 406 to align the fields of view of each component. The tube lens 404 and the objective lens 406 are thus able to facilitate, through the camera 402, the capture images of a target organism (e.g., C. elegans) within the first plate 150. Thus, the imaging system 400 forms a microscopic imaging system.

The imaging system 400 further includes one or more light sources 408a, 408b. For example, the light sources 408a, 408b may be LED light sources producing a specific wavelength or range of wavelengths of light. The light sources 408a, 408b are directed towards the field of view of the camera 402, which may include the bottom of a plate under observation (e.g., the first plate 150 in the first plate station 310).

The light sources 408a, 408b of the imaging system 400 are directed through one or more filter cubes 410a, 410b. The filter cubes 410a, 410b may include one or more optical filters and/or mirrors configured to redirect and filter light to and from the plate under observation and the camera capturing images. For example, the filter cubes 410a, 410b may include an excitation filter configured to filter light from the light sources 408a, 408b within an excitation wavelength (e.g., an excitation wavelength matching that of a dye or fluorescent protein disposed within the first plate 150 and the organisms contained therein). The filter cubes 410a, 410b may further include an emission filter configured to block residual excitation light and selectively transmit the fluorescence emitted by the sample (e.g., by the organisms within the plate).

### Exemplary Control System and Operation

As shown in FIG. 1, the controller 500 is in communication (e.g., wired or wireless communication) with each of the robotic arm 100, the organism manipulator 120, the imaging system 400, and the actuators that move the carriage 340. The controller 500 is configured to actuate the robotic arm 100 (e.g., based on instructions stored in a memory thereof) to perform a movement or operation. Furthermore, the controller 500 is configured to actuate the end effector plate 106 to rotate one of the end effectors into the active position. The controller 500 is configured to operate any one of the elements of the organism manipulator 120 (e.g., via commination with the electronics box 126 thereof).

The controller 500 is further configured to actuate the carriage 340 to move the imaging system 400 to a desired position. The controller 500 is configured to operate any one of the elements of the imaging system 400, including the camera 402 thereof to collect image data. The controller 500 is further configured to process (e.g., via a processor thereof) information from the various elements of the system and actuate other elements of the system 10 based on such information. For example, visual data from the camera 402 may be used to adjust the location of the wire-loop picking tool 130.

In one configuration, the circuits of the controller 500 are in the form of machine or computer-readable media that is executable by a processor. As described herein, the machine-readable media facilitates the performance of certain operations to enable the reception and transmission of data. For example, the machine-readable media may provide an instruction (e.g., command, etc.) to acquire data. In this regard, the machine-readable media may include programmable logic that defines the frequency of acquisition of the data (or, transmission of the data). The computer-readable media may include code written in any programming language. The computer-readable program code may be executed on one processor, multiple co-located processors, multiple remote processors, or any combination of local and remote processors. Remote processors may be connected to each other through any type of network (e.g., CAN bus, etc.).

In another configuration, the circuits of the controller 500 are implemented as hardware units, such as electronic control units. As such, the circuits of the controller 500 may be implemented as one or more circuitry components including, but not limited to, processing circuitry, network interfaces, peripheral devices, input devices, output devices, sensors, etc. In some implementations, the circuits of the controller 500 may take the form of one or more analog circuits, electronic circuits (e.g., integrated circuits (IC), discrete circuits, system on a chip (SOCs) circuits, microcontrollers, etc.), telecommunication circuits, hybrid circuits, and any other type of “circuit.” In this regard, the circuits of the controller 500 may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on). The circuits of the controller 500 may also include programmable hardware devices such as field programmable gate arrays, programmable array logic, programmable logic devices or the like. The circuits of the controller 500 may include one or more memory devices for storing instructions that are executable by the processor(s) of the circuits of the controller 500. The one or more memory devices and processor(s) may have the same definition as provided below with respect to the memory device and processor. In some hardware unit configurations, the circuits of the controller 500 may be geographically dispersed throughout separate locations. Alternatively and as shown, the circuits of the controller 500 may be implemented in or within a single unit/housing, which is shown as the controller 500.

In the example shown, the controller 500 includes a processing circuit having the processor and the memory device. The processing circuit may be structured or configured to execute or implement the instructions, commands, and/or control processes described herein with respect to the circuits of the controller 500. The depicted configuration represents the circuits of the control system as machine or computer-readable media. However, as mentioned above, this illustration is not meant to be limiting as the present disclosure contemplates other implementations where the circuits of the controller 500 or at least one circuit of the circuits of the controller 500, is configured as a hardware unit. All such combinations and variations are intended to fall within the scope of the present disclosure.

The hardware and data processing components used to implement the various processes, operations, illustrative logics, logical blocks, modules and circuits described in connection with the implementations disclosed herein (e.g., the processor) may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some implementations, the one or more processors may be shared by multiple circuits (e.g., the circuits of the controller 500 may comprise or otherwise share the same processor which, in some example implementations, may execute instructions stored, or otherwise accessed, via different areas of memory). Alternatively or additionally, the one or more processors may be structured to perform or otherwise execute certain operations independent of one or more co-processors. In other example implementations, two or more processors may be coupled via a bus to enable independent, parallel, pipelined, or multi-threaded instruction execution. All such variations are intended to fall within the scope of the present disclosure.

The memory device (e.g., memory, memory unit, storage device) may include one or more devices (e.g., RAM, ROM, Flash memory, hard disk storage) for storing data and/or computer code for completing or facilitating the various processes, layers, and modules described in the present disclosure. The memory device may be communicably connected to the processor to provide computer code or instructions to the processor for executing at least some of the processes described herein. Moreover, the memory device may be or include tangible, non-transient volatile memory or non-volatile memory. Accordingly, the memory device may include database components, object code components, script components, or any other type of information structure for supporting the various activities and information structures described herein.

### Exemplary Method of Use

The system 10 may be used to transport and organize the target organisms, such as in a genetic testing and/or manipulation study. Rather than a laborious manual process, the system 10 can be implemented in an automated fashion to pick plates, identify organisms, move target organisms, and re-distribute plates in an organized manner.

In use, the first shelf array 200 may include a plurality of plates and/or lids, including plate 150. Each of the plates may be a petri dish including agar media and target organisms therein (e.g., C. elegans). The controller 500 may include a memory that stores location data for the plurality of plates. For example, characteristics of the group of organisms in a given plate may be stored in the memory. Then, the robotic arm 100 may be actuated (e.g., via the controller 500) to pick a plate (e.g., plate 150) from the first shelf array 200. The plate manipulation operation is described with reference to FIGS. 5A and 5B. The plate 150 is then deposited onto the first plate station 310 of the working platform 300.

Once the plate 150 is in plate on the working platform 300, the robotic arm 100 rotates the end effector plate 106 so that the plate manipulator 110 is in the inactive position and the organism manipulator 120 is in the active position. Furthermore, the imaging system 400 may be moved (e.g., via instructions from the controller 500) so that the camera 402 can view the bottom of the plate 150.

The camera 122 of the organism manipulator 120 may be activated to view the contents of the first plate 150 from the top side. Data from the camera 122 may be relayed to the controller 500 or a controller local to the electronics box 126. Furthermore, the camera 402 may be activated to view the contents of the first plate 150 from the bottom side. Data from the camera 402 may be relayed to the controller 500 of a controller local to the imaging system 400.

Either the camera 122 or the camera 402 is used to view the target organisms in the first plate 150 and to identify organisms with a target characteristic. For example, organisms with a specific physical feature or phenotype may be identified. This identification process may be first aided by a fluoroscopic imaging system in the imaging system 400, and then the physical manipulation process may be completed by the organism manipulator 120.

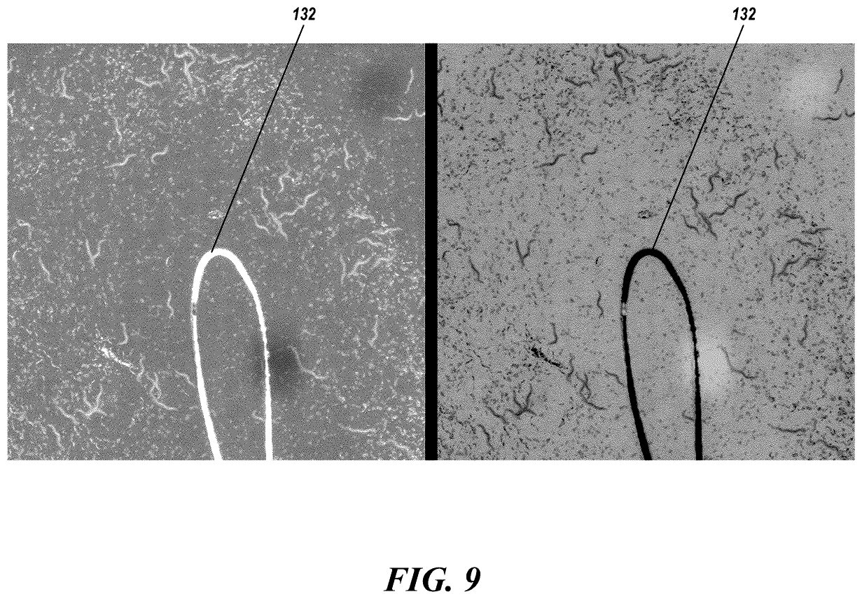

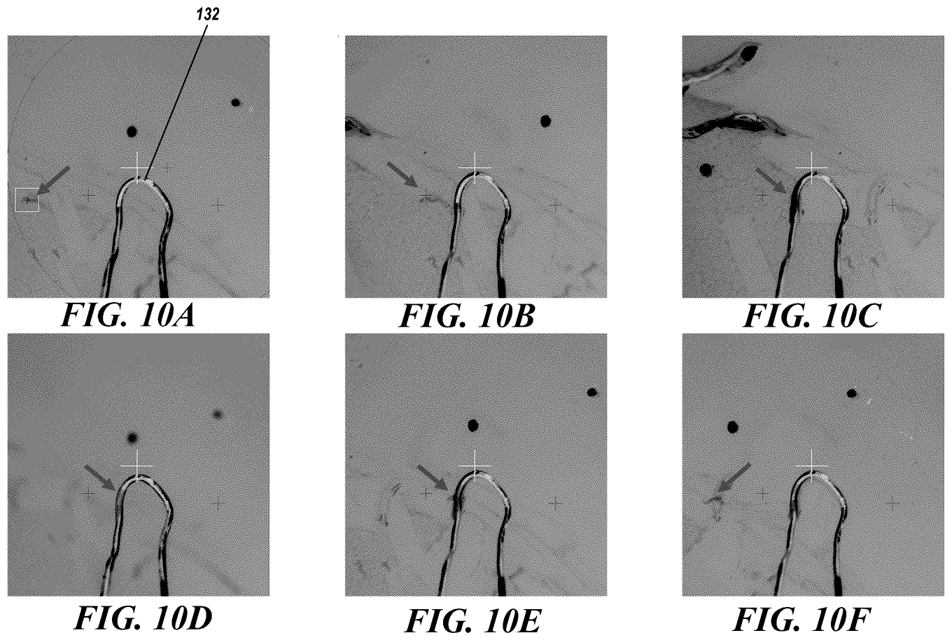

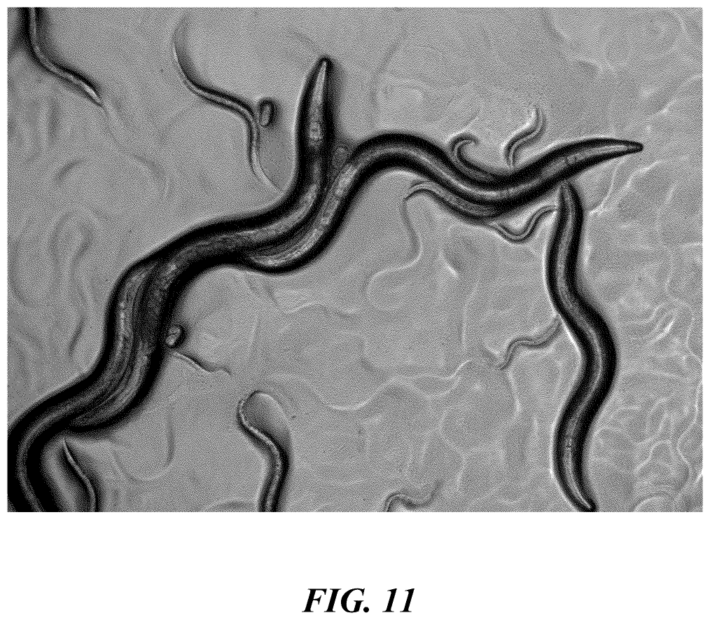

Once a target organism is identified, the system 10 can pick and move that organism (e.g., to another plate disposed on the working platform 300). As shown in FIG. 9, the wire loop 132 is visible against a field of C. elegans as the target organisms in the plate 150. FIGS. 10A-10F show a sequence of moving the wire loop 132 to pick, move, and release a single C. elegans worm using the camera 122. As shown, the target organisms' positions in the images are shown by red arrows. In FIG. 10A, a worm is being tracked by machine vision. In FIG. 10B, the worm is centered at the picking spot. In FIG. 10C, the wire loop 132 is swiping and contacting the worm. In FIG. 10D, the wire loop 132 is raised up and moving above the agar. In FIG. 10E, to put down the worm, the wire loop 132 is lowered and touching the agar. Finally, in FIG. 10F, the wire loop 132 is swiping to release the worm. FIG. 11 shows a close-up, high-resolution image of the worms (C. elegans) taken from the camera 402 of the imaging system 400.

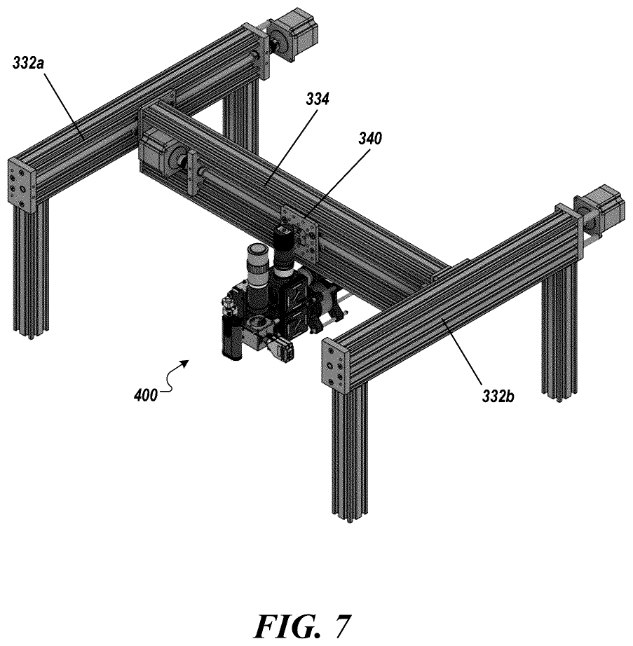

The system 10 can perform several worm identification and picking operations. For example, the system 10 can identify and relocate all of a certain type of organisms within a single plate. Furthermore, the system 10 can store the plate in the first shelf array 200 and retain information about its contents and location for later use. Overall, the system 10 can perform a variety of organism organization operations in an automated process, creating a more efficient system for researchers. The system 10 may be used in various additional processes and with various alternative structures and features. The structures and features shown in FIGS. 1-11 are exemplary only and do not limit the disclosure. Any combination of features or any alternative features described herein or in the experimental section that follows is contemplated by this disclosure.

### Experimental Study and Results

A study was conducted to construct and evaluate a general-purpose robotic system allowing automated phenotyping and genetic manipulation of C. elegans on agar substrates, using techniques resembling manual methods.

The nematode Caenorhabditis elegans is one of the most widely studied organisms in biology due to its small size, rapid life cycle, and manipulable genetics. Research with C. elegans depends on labor-intensive and time-consuming manual procedures, imposing a major bottleneck for many studies, especially for those involving large numbers of animals. This study describes a general-purpose tool, WormPicker, a robotic system capable of performing complex genetic manipulations and other tasks by imaging, phenotyping, and transferring C. elegans on standard agar media. The system uses a motorized stage to move an imaging system and a robotic arm over an array of agar plates. Machine vision tools identify animals and assay developmental stage, morphology, sex, expression of fluorescent reporters, and other phenotypes. Based on the results of these assays, the robotic arm selectively transfers individual animals using an electrically self-sterilized wire loop, with the aid of machine vision and electrical capacitance sensing. Automated C. elegans manipulation shows reliability and throughput comparable with standard manual methods. The study developed software to enable the system to autonomously carry out complex protocols. To validate the effectiveness and versatility of our methods, the study used the system to perform a collection of common C. elegans procedures, including genetic crossing, genetic mapping, and genomic integration of a transgene. The robotic system accelerates C. elegans research and opens possibilities for performing genetic and pharmacological screens that would be impractical using manual methods.

### Introduction

Classical genetics, which investigates the heritability of traits across generations, usually requires manipulating the reproductive behaviors of organisms and inferring their genetic properties by assaying their traits. The microscopic nematode Caenorhabditis elegans is one of the most widely used genetic models in life sciences due to its easy maintenance, optical transparency, and rapid life cycle. Studies in C. elegans have pioneered major fundamental advances in biology, including those in programmed cell death, aging, RNA interference, and axon guidance. Work with worms has pioneered important techniques in modern biology including genome sequencing, cell lineage tracing, gene editing, electron microscopic reconstruction of neural connectivity, demonstration of green fluorescent protein, and optogenetic manipulation of neural activity. Genetic manipulations in C. elegans are performed by manual procedures, which involve the identification of animals under a microscope and the transfer of worms or embryos from one agar plate to another using a wire pick. While manual procedures are reliable and technically simple, they have important limitations. Manual methods are labor-intensive since they require animals to be manipulated individually. This presents challenges to experiments that require thousands of groups to be managed, for example conducting genetic screen, working with collections of wild isolates, or dealing with mutagenized strains of the Million Mutation Project.

During the design of lab experiments, the use of manual procedures creates practical limits to the number of conditions and the number of replicates for each condition, weakening statistical power. Standard population sizes used for C. elegans lifespan experiments have been shown to be underpowered for moderate differences in lifespan between groups. Finally, manual approaches require training and are prone to errors. This reliance on investigator-learned skills imposes a barrier to entry for scientists without C. elegans experience wishing to use this system.

This study presents WormPicker, a general-purpose robotic system allowing automated phenotyping and genetic manipulation of C. elegans on agar substrates, using techniques resembling manual methods. The exemplary device contains a 3D motorized stage carrying a robotic arm and an optical system. The robotic arm manipulates animals using a thin, electrically sterilized platinum wire loop. Analogous to manual methods, the robotic arm picks worms by performing spatially and temporally controlled motions above and on the agar surface, using food bacteria to encourage the worm to adhere to the loop. Contact between the platinum wire loop and the agar surface is perceived by a capacitive touch sensing circuit, providing feedback in conjunction with the imaging system for fine adjustment of the pick trajectory relative to the animal. The robot's optical system is capable of monitoring animals over an entire plate at low magnification [6-cm diameter circular field of view (FOV)] while simultaneously imaging individual animals at high magnification (1.88 mm×1.57 mm FOV) to obtain more detailed morphological and/or fluorescence information.

Using machine vision methods, worms can be recognized and tracked over the plates in low magnification and undergo detailed phenotyping in high magnification across different attributes, including developmental stage, morphology, sex, and fluorescence expression. The study developed system control software through which the user can specify multistep genetic procedures to be performed. Using these automated tools, the study successfully carried out three genetic procedures commonly performed in C. elegans research. First, the study generated a genetic cross between transgenic and mutant animals using a classic genetic hybridization scheme. Second, the study performed genetic mapping of a genome-integrated fluorescent transgene. Finally, the study integrated an extrachromosomal transgenic array into the genome, creating stable transgenic lines. Successful completion of these complex genetic procedures demonstrates WormPicker's effectiveness and versatility as a broadly useful tool for C. elegans genetics.

### Overview of System Design

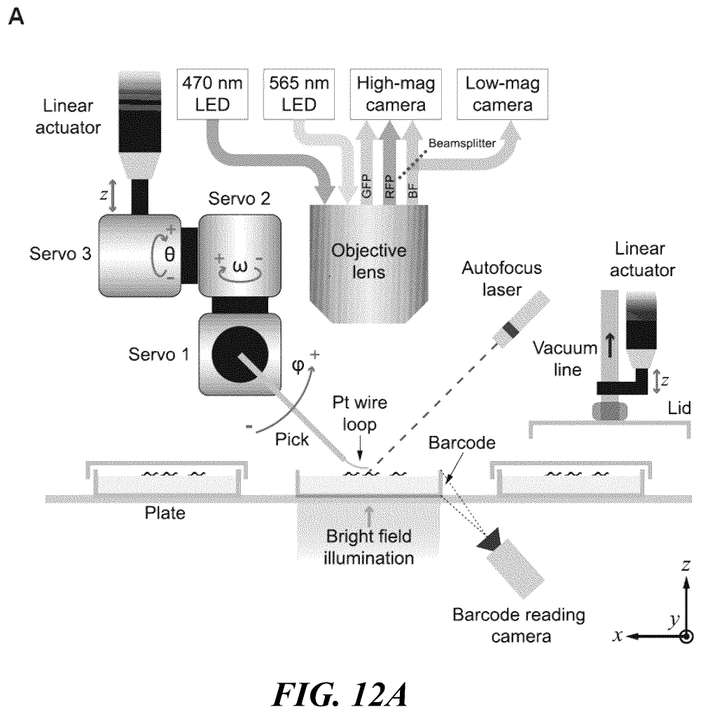

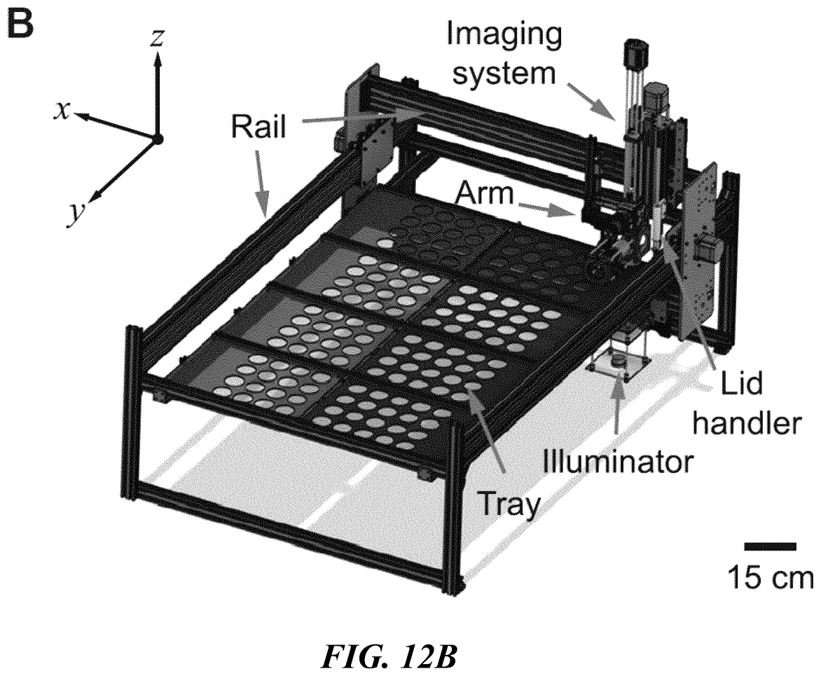

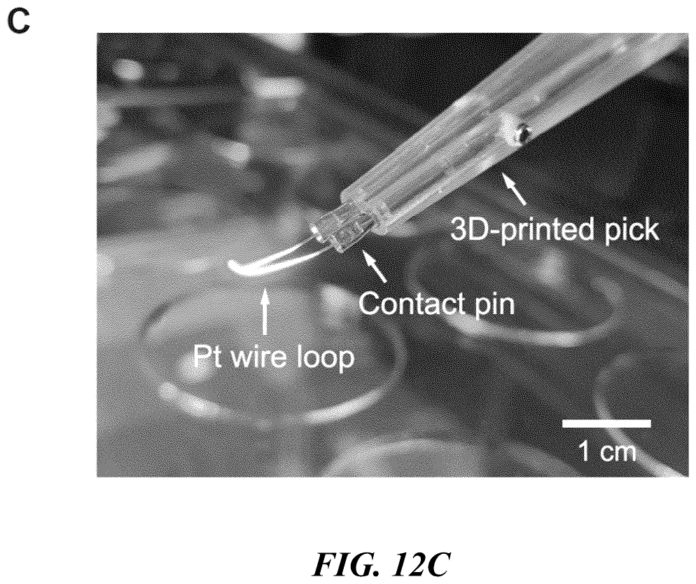

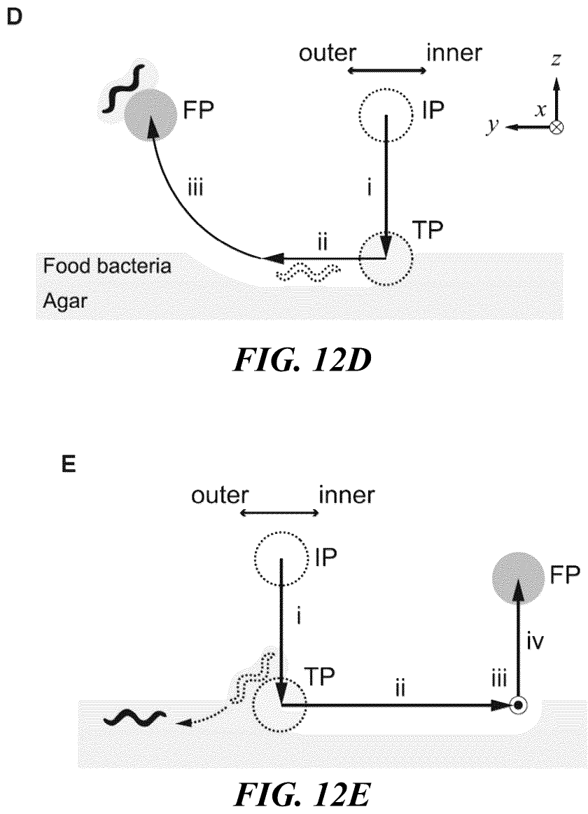

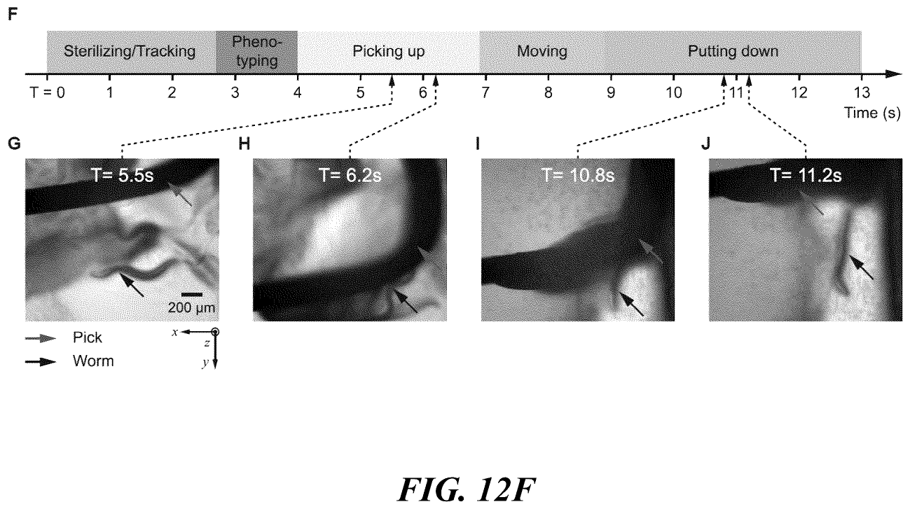

The exemplary system contains a robotic picking arm, optical imaging system, lid manipulators, and other elements mounted on a 3D motorized stage to work with an array of up to 144 agar plates, as shown in FIGS. 12A and 12B. The array of plates is housed on a platform, as shown in FIG. 12B, containing eight trays that can be prepared separately and then slid into the automated system. Each plate on the tray can be labeled with an adhesive barcode and text. The study developed software to track plates during experiments using their barcode identifiers. The imaging system is designed for bright field and fluorescence imaging through low- (0.035×) and high-magnification (10×) optical pathways. The low-magnification stream has a 6-cm diameter circular FOV, capable of imaging one 6-cm diameter plate; the high-magnification stream has a 1.88 mm×1.57 mm FOV, capable of imaging individual animals in detail. For bright field imaging, light is collected by an objective lens and divided into low- and high-magnification imaging streams by a beamsplitter. For #uorescence imaging, collimated excitation LEDs (center wavelengths 470 and 565 nm) and fluorescence optics enable imaging in green (503- to 541-nm wavelength) and red (606- to 661-nm wavelength) channels via the high-magnification pathway.

To quickly bring the surface of a plate into focus, the exemplary system uses a laser-based autofocusing system, as shown in FIG. 12A. A 532-nm laser pointer with an integrated cylindrical lens generates a line projected onto the agar surface from an oblique angle, providing feedback for the motorized stage to move the imaging system to the correct position. Plate lids are manipulated by a vacuum gripping system. Two lid handlers are mounted to either side of the main motorized gantry for manipulating lids for a source plate and a destination plate separately. Each vacuum lid gripper is raised and lowered by a motorized linear actuator. The robotic picking arm contains a motorized linear actuator for fine adjustment of the pick's height and three servo motors to provide rotational degrees of freedom. The 3D-printed pick contains a platinum wire loop attached to its end, as shown in FIG. 12C, for manipulating C. elegans in a manner analogous to manual methods.

Conventional pick sterilization prior to manipulating C. elegans on agar substrates requires an open flame, which poses safety risks in an automated system. This study adopted an electric sterilization approach in which a current is passed through the wire loop to sterilize it via resistive heating. Picking up C. elegans requires very fine control of the pick to avoid damage to the worm or the agar surface. While the horizontal position of the pick can be monitored by the imaging system, its height relative to the agar surface is more difficult to determine. To address this problem, the study developed a capacitive touch-sensing circuit that detects contact between the platinum wire and agar surface and provides feedback for fine tuning the pick's movements. Additionally, the pick's height relative to the optimal focus of the imaging system can be monitored by measuring the intensity of the object. As shown in FIG. 12D, to pick up a worm, the wire pick is positioned above the agar [initial position (IP)], with a horizontal offset to the target worm. The linear actuator lowers the pick until contacting the agar surface [touching position (TP)] as perceived by the capacitive touch sensor (phase i). Next, Servo 2 horizontally swipes the pick on the agar surface (phase ii). Servos 1 and 3 act simultaneously to perform a curved motion (phase iii) for picking up the target animal using the outer side of the wire loop. As shown in FIG. 12E, to release the worm from the pick to the substrate, the pick is positioned above the agar (IP); the linear actuator vertically lowers (phase i) the pick until it touches the agar (TP); the Servo 2 horizontally swipes (phase ii) the pick on the agar surface, to release the worm from the pick. The basic pick-up and put-down actions are chained in series for automated C. elegans transfer, as shown in FIG. 12F.

### Machine Vision Enables Automated Identification and Phenotyping

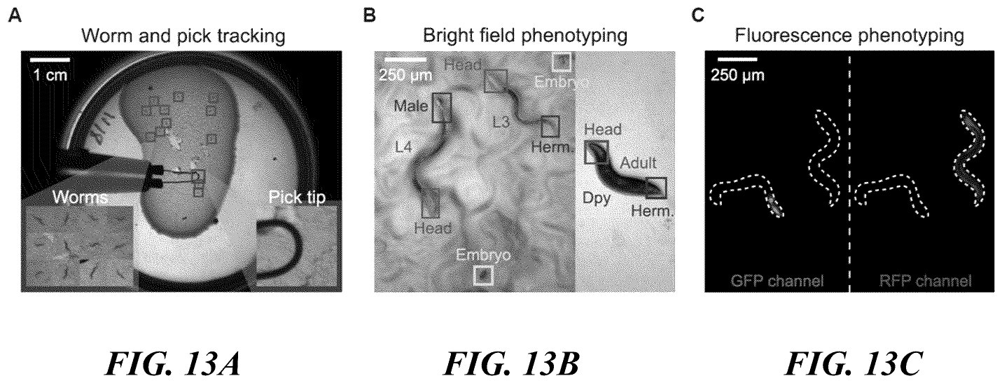

The study developed machine vision analysis software for the low- and high-magnification imaging streams. The study analyzed the low-magnification images, as shown in FIG. 13A, using a combination of convolutional neural networks (CNNs) and motion detection for tracking animals and the pick in real time. High-magnification bright field images, as shown in FIG. 13B, were analyzed by a set of Mask-Regional CNNs (Mask-RCNNs) capable of performing pixel-wise object segmentations. Animals' contour geometries are analyzed for developmental stage and morphology. In addition, the study trained separate networks for sex determination and embryo detection. Inferences from multiple Mask-RCNNs are integrated for assaying phenotypes for individual animals over different attributes.

For high-magnification fluorescence images, as shown in FIG. 13C, the study performed intensity analysis to extract the valid fluorescent signals from the background. To assay fluorescence expression, the fluorescence images were correlated with the animal contours segmented using the bright field images.

### WormPicker Reliably Picks C. Elegans of Various Stages and Phenotypes

First, the study asked whether the automated picking causes damage to C. elegans. The system was used to pick animals of all stages, ranging from L1 larvae to day 5 adults, and an array of mutants, including lin-15 (multivulva), rol-6 (roller), unc-13 (uncoordinated and paralyzed), and lpr-1 (fragile cuticles) (32). The study measured the number alive 24 h after the automated picking. As a control, the study repeated the procedure using the standard manual methods. The study observed that animals manipulated by WormPicker showed viability comparable with that of standard methods. The study asked how effectively the system picks up and puts down animals. The study manually verified the success of individual pick-up and put-down attempts through the live image stream. The study observed success rates ˜90% for picking up and putting down different types of animals. When working with unseeded plates, the study precoated the wire loop with bacteria and observed similar success rates as for seeded plates. These experiments show that WormPicker is safe and effective for transferring many different types of C. elegans, including young, aged animals, and various mutants.

To compare the rate of automated and manual picking, the study evaluated how quickly the robot and human researchers could perform a fluorescent animal sorting task. The study used a strain in which some but not all worms carry a myo-2::GFP-labeled fluorescent extrachromosomal array (YX256); the task was to sort these worms into two plates containing fluorescent and non-fluorescent animals. The robotic system sorted the animals (of mixed stages) with a throughput of 3.21±0.66 (mean±SD) animals per minute (APM). We recruited a group of C. elegans researchers (N=21) to perform the same task using standard methods. The mean and median years of their C. elegans experience were 7.61 and 5 years, respectively. The study tasked each volunteer to sort ˜20 animals (of mixed stages) under a fluorescence stereoscope. Both WormPicker and the researchers picked individual worms and sterilized the pick between transfers. The manual picking throughput was 3.56±1.67 (mean±SD) APM.

These results show that the throughput of the robotic system for this fluorescent animal sorting task is comparable with that of experienced human researchers. However, in other implementations such as that of FIGS. 1-8, the system outpaces human researchers.

The automated system maintains an aseptic environment—As in manual work, it is important to minimize contamination of media in our automated system. The study designed the WormPicker to maintain an aseptic environment. The study built the system inside a panel enclosure to prevent airborne contaminants from entering. The study sanitized the active components, including the robotic arm, microscope, and plate trays using 70% ethanol before experiments. During experiments, plates had lids on for most of the time, except briefly during picking operations. The study did not observe any plate contaminated 10 days after being manipulated by WormPicker (N=101 plates). In comparison, 3.4% of the plates were contaminated after manual picking in the same room (N=119 plates). These results show that our protocols were sufficient for maintaining an aseptic environment for the experiments.

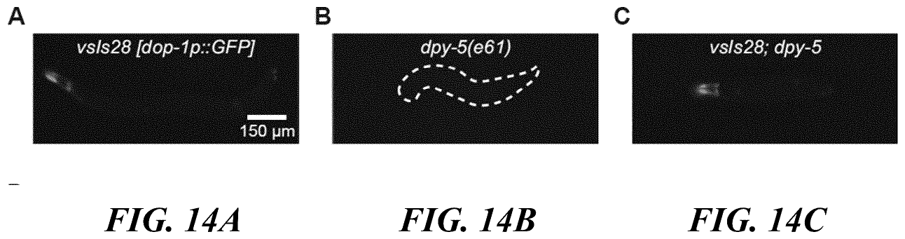

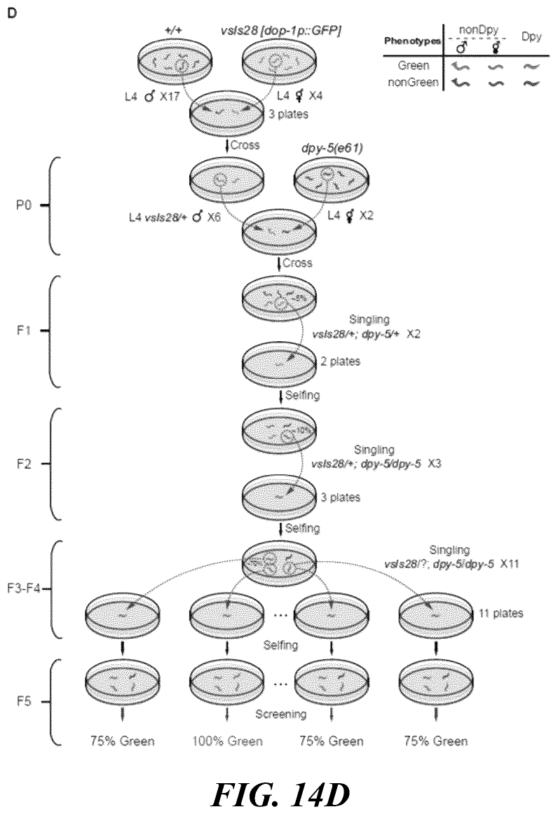

Scripting toolsets enable complex genetic manipulations—In order for WormPicker to be useful for practical laboratory work, the basic elements of identifying and transferring worms need to be combined to form complex genetics procedures. To that end, The study developed system control software, WormPickerControl. An application programming interface (API) enables the user to specify C. elegans procedures to be carried out by the automated system. The study developed a library of source scripts, each responsible for a specific task ranging from simple to complex. The automated system catalogs a set of plates based on their barcode identifier and stores their information in a database. Using WormPickerControl, The study developed and tested a collection of genetic procedures commonly undertaken in C. elegans labs (FIGS. 14A-16). These are not intended to be a complete set of genetic protocols but rather to provide a useful framework that can be modified and adapted to other experiments as needed.

Automated genetic cross—The genetic cross, by which two or more mutations or transgenes are combined, is performed by placing males together with hermaphrodites on the same agar plate. While monitoring the plates, researchers pick cross-progeny with desired phenotypes. Most genetic crosses require manipulating animals over multiple generations. The study automated a genetic cross between two C. elegans strains: dop-1p::GFP fluorescent transgenic (LX831) and a dpy-5(e61) mutant (CB61). The LX831 strain contains an integrated transgene vsIs28 [dop-1p::GFP] and has a Green phenotype; i.e. GFP is expressed in several cell types including some neurons and muscles, as shown in FIG. 14A. The dpy-5 mutant has a Dumpy (Dpy) phenotype, characterized by a morphology that is shorter and stouter than wild-type animals, as shown in FIG. 14B. The vsIs28 transgene is dominant (i.e. both heterozygotes and homozygotes show green fluorescence) whereas the dpy-5 mutation is recessive (i.e. only the homozygotes show the Dpy phenotype). The resulting hybridized strain displays both phenotypes of two parental strains; i.e. it is both Green and Dpy, as shown in FIG. 14C.

FIG. 14D depicts a schematic of the genetic cross. WormPicker picked 17 L4 males from a wild-type (N2) population that consisted of a mixture of males and hermaphrodites. It also picked four L4 hermaphrodites from the LX831 strain to the same plates. Green males were visible in the progeny, indicating that the mating was successful. Next, six Green L4 vsIs28 heterozygous males and two dpy-5 homozygous L4 hermaphrodites were picked onto a plate for mating. The study considered this mating the parental (P0) generation. Filial generation 1 (F1) progeny included animals that were Green and not Dpy. These were the desired worms heterozygous for both vsIs28 and dpy-5 and were therefore transferred by WormPicker to fresh plates. The automated system screened over the plate and singled two F1 hermaphrodites with the wanted phenotypes. Four phenotypes were observed in the F2 generation, including Dpy-Green, Dpy-nonGreen, nonDpy-Green, and nonDpy-nonGreen. The frequency of the desired Dpy-Green phenotype was ˜10%. WormPicker inspected the F2 populations and singled three Dpy-Green animals. F3 populations descending from each of the three F2s were 100% Dpy but only 75% Green indicating that they were homozygous for dpy-5 but heterozygous for vsIs28. WormPicker then singled 11 Dpy-Green animals. The automated system screened for the percentage of Green F5s descending from each of the eleven F4s and identified one line displaying homozygous fluorescent; i.e. 100% of F5s were Green. The results were verified by an experienced C. elegans researcher by noting that the resulting strain was positive for both Green and Dpy phenotypes and that these phenotypes bred true in subsequent generations.

### Automated Genetic Mapping of a Transgene

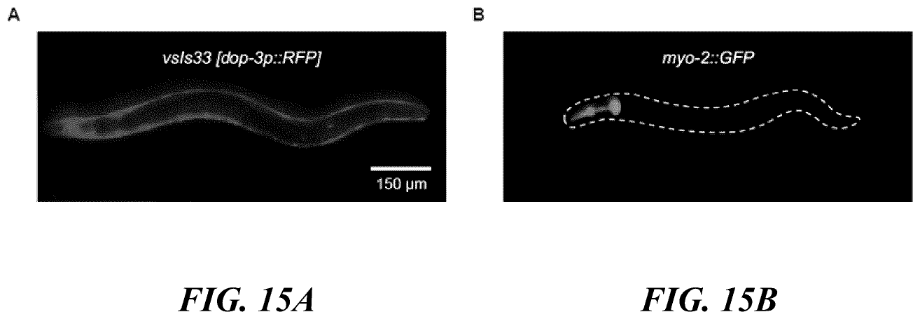

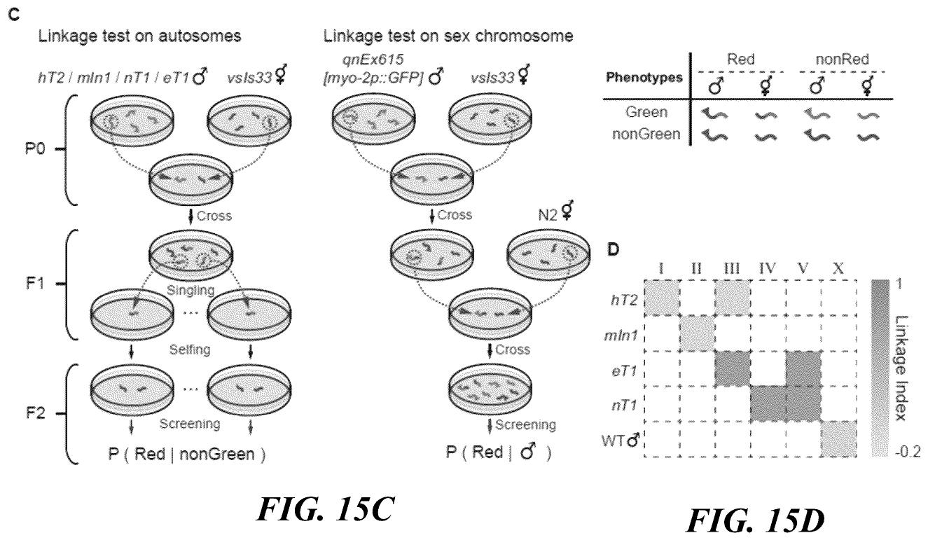

The identification of the genotype causing a particular phenotype usually requires genetic linkage analysis. The first step in such analysis is to identify the chromosomes harboring the genetic change. C. elegans has six chromosomes, of which five are autosomes and one is the X chromosome. Hermaphrodites are diploid for all six chromosomes, while males are diploid for five autosomes and haploid for the X chromosome. Genetic mapping in C. elegans can be performed by setting up genetic crosses between the strain of interest and a set of marker strains and measuring the segregation pattern between the marker phenotypes and the phenotype of interest. The study used WormPicker to perform an automated genetic mapping of an integrated red fluorescent transgene vsIs33 [dop-3p::RFP] (LX811). This strain has a Red phenotype; i.e. RFP is expressed in cells expressing DOP-3 dopamine receptors, as shown in FIG. 15A. The study evaluated the linkage of vsIs33 to autosomes by setting up crosses between our strain of interest (LX811) and a set of balancer strains. These balancer strains are labeled by myo-2::GFP markers and have a Green phenotype (GFP expressed in pharyngeal muscles), as shown in FIG. 15B. The study used the reciprocal translocations hT2 [qIs48] (I; III), eT1 [umnIs12] (III; V), and nT1 [qIs51] (IV; V) to test for linkage to large portions of chromosomes I and III, III and V, and IV and V, respectively; the study also used the inversion mIn1 (40) [mIs14] (II), to test for linkage to chromosome II, as shown in FIG. 15C.

WormPicker picked L4 Green males from the balancer strains and hermaphrodites from the strain of interest (LX811) for mating. WormPicker then screened for F1 Red-Green hermaphrodites which were subsequently singled. The double-fluorescent F1 hermaphrodites self-fertilized and produced F2s, where the percentages of Red among nonGreen animals were assessed to test for linkage between vsIs33 and particular autosomes. According to the classic genetic theory, linkage of the transgene to the tested balancer chromosomes would yield 100% of nonGreen animals to be Red, whereas nonlinkage of the transgene to the balancer would be reflected by 75% Red among the nonGreen progeny. For testing linkage to the X chromosome, WormPicker generated a genetic cross between LX811 hermaphrodites and wild-type males harboring a dominant extrachromosomal transgene qnEx615 [myo-2p::GFP] (NQ1155), which helped us to identify F1 cross-progeny. As shown in FIG. 15C, F1 Red-Green males were picked out for mating with N2 (wild-type) hermaphrodites. F2s were screened by the robotic system, and the observed percentage of Red among males was used for quantifying the degree to which vsIs33 is linked to the X chromosome. The theory predicts that none of the F2 males would be Red if X-linked and that 50% of the F2 males would be Red if not X-linked. To quantify the strength of linkage, the study defined a Linkage Index ranging from −1 to 1, where 1 implies the strongest possible linkage and 0 the weakest. The data, as shown in FIG. 15D, indicate a strong linkage of vsIs33 both to eT1 and nT1, and no linkage to hT2, to mIn1, or to the X chromosome. Since both eT1 and nT1 balance a large portion of chromosome V, our results imply that the RFP transgene is located on chromosome V.

### Genomic Integration of a Transgene

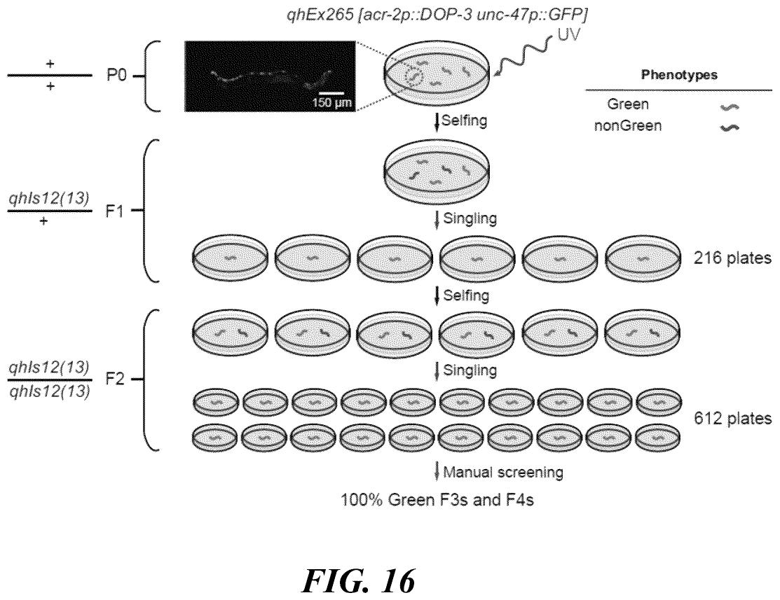

Transgenic C. elegans are usually generated by microinjection, through which cloned DNAs are delivered to the distal gonadal arm, forming extrachromosomal transgenic arrays. To provide stable inheritance and expression, the transgenic arrays can be integrated to the genome. A proven method for genomic integration is to irradiate the strain of interest, causing breakage in chromosomes, which triggers DNA repair, a process through which the transgenic arrays can be ligated to the chromosomes by chance. Due to its low frequency, identifying animals with the transgene integrated requires isolating at least several hundred individual worms and screening for 100% inheritance of the transgene in subsequent generations, a highly labor-intensive task (43). In particular, the need to single hundreds of animals can consume a substantial amount of time, even for experienced C. elegans researchers. Using WormPicker, the study performed a genomic integration of an acr-2p::DOP-3 extrachromosomal array labeled by a green fluorescent marker unc-47p::GFP (YX293), as shown in FIG. 16. The strain displayed ˜65% transmission of the transgenic array to the next generation. To create the P0 worms, we irradiated 68 L4s with ultraviolet light (254 nm, 30 mJ/cm2). WormPicker isolated 216 F1s to individual plates. From these plates, the automated system isolated 612 of their progenies (F2s) to individual plates. The F2 animals were manually screened for 100% transmission of the transgene to F3s and F4s. Among these, two lines were identified as potential independent integrants. For both lines, 100% transmission of the transgene was confirmed over several subsequent generations, consistent with successful array integration.

### Discussion

In this work, the study demonstrated that WormPicker can automate a variety of C. elegans genetic procedures usually performed by manual methods. In addition, our scripting tools provide flexibility for carrying out customized experiments as well as integrating the system into diverse genetic screens and analyses, potentially including pharmacological screening, screening for aging phenotypes, and studies of natural genetic variation. Robotic manipulation of C. elegans opens possibilities for experiments that would be difficult or impractical for manual methods, especially for those involving a large number of strains or conditions. For example, our laboratory is using this machine to perform a genetic screen for modifiers of stress-induced sleep. The deep-learning-aided machine vision methods that the study developed here, capable of segmenting individual animals and assaying them across different attributes, may find other applications for C. elegans studies, for example, in analyses of locomotion, aging, and sleep. The self-sterilizing loop design can be used for automatically manipulating other microscopic organisms, for example, other nematodes, Drosophila larvae, bacteria, and fungi.

The study demonstrated that WormPicker's machine vision is able to segment individual animals on highly populated plates. For the experiments presented here, the study programmed the robot to perform intermediate picking before transferring animals to their destinations, as a method of handling clusters of worms. In addition, brief blue light illumination or plate vibration could be used to disperse clusters of animals. Although WormPicker's machine vision system works well for recognizing and tracking the wild-type animals and mutants tested here, some modifications of our algorithms may be necessary for some mutants with unusual morphologies and behaviors.

### Materials and Methods

In the exemplary system of this study, WormPicker is based on a 1.5 m×1 m rectangular framing system constructed from aluminum extrusions (OpenBuilds V-Slot 20 mm×20 mm, 20 mm×40 mm, and C-Beam 40 mm×80 mm). WormPicker's imaging system and picking arm are moved by a motorized stage along three axes: X (80 cm travel), Y (125 cm), and Z (30 cm). In addition, there is a linear carriage under the plate tray that moves the illumination and plate tracking system. All axes are driven by stepper motors (NEMA 23, 1.8° per step). For the X and Y axes, motors mounted to the carriage drive motion via a belt attached to both ends of the rail. For the Z axis, a stepper motor drives motion through the rotation of a lead screw. The maximum speed of the X axis was set to 146.67 mm/s, Y axis 146.67 mm/s, and Z axis 8.33 mm/s. The maximum acceleration of the X axis was set to 120 mm/s², the Y axis 120 mm/s², and the Z axis 10 mm/s². Stepper motors are controlled through a PC via an OpenBuilds BlackBox motion control system using GRBL firmware. All aspects of the robotic system were controlled by an Origin PC with an Intel® Core i9-10900K CPU at 3.7 GHz and 64 GB of RAM, running Windows 10.

### Dual-Magnification Multimodal Optical Imaging System

To implement bright field imaging, the study constructed an illumination system under the platform, in which light from a white LED is diffused by a ground glass and approximately collimated by a Fresnel lens. The low- and high-magnification imaging paths share a common objective lens (achromatic doublet, focal length 100 mm). Light is collected by the objective lens and then divided into the low- and the high-magnification imaging streams by a beamsplitter (ratio of transmission: reflection is 90%:10%). The low-magnification image is relayed to a machine vision camera by a camera lens, while the high-magnification image is formed at a CMOS camera through a set of teleconverter lenses and a tube lens. The high-magnification pathway is an infinity-corrected microscopy system that can support both bright field and fluorescence imaging.

For fluorescence imaging, two collimated excitation LEDs (center wavelengths 470 and 565 nm) and a set of dual-band fluorescence optics (Chroma 59022) were built in the infinity space in the high-magnification pathway. The spectral characteristics of the fluorescence optics were selected to enable both GFP and RFP imaging. Switching between the imaging modes can be achieved by digital relay circuits controlling the excitation LEDs and the underplatform illumination.

### Robotic Picking Arm

The study built a robotic arm for manipulating C. elegans on agar media using a wire loop. The robotic picking assembly consists of a linear actuator, three servo motors, and a 3D-printed worm pick. The linear actuator fine-tunes the z height of the picking arm through a linear carriage. The servo motors are chained orthogonally to provide 3 degrees of freedom in rotation for the worm pick. The 3D-printed worm pick is mounted to Servo 1. The design allows two copper wires to fit into the pick stem from the proximal end, and these two wires are connected by a portion of the looped platinum wire (90% Pt, 10% Ir, 254 μm diameter) at the distal end of the pick. The platinum wire is crimped to copper contact pins for attaching to the end of the pick.

To sterilize the pick, the platinum wire loop is connected to a 5-V DC power supply (Fig. S3G). The resulting 4.5-A DC current sent through the wire heats the loop to a temperature we estimate to exceed 1,000° C. based on its color. The study used the wire loop as a capacitive sensing probe to monitor contact between the wire and the agar surface and to provide feedback for picking trajectories. For the sensing circuit to function properly, the platinum wire loop is first disconnected from the heating circuit. The capacitance change due to the contact is sensed by a capacitive touch sensor (SparkFun, AT42QT1011), the voltage output of which is monitored by a data acquisition device (LabJack).

Pick Motion Trajectories for Manipulating C. elegans

To pick up an animal, the wire pick is positioned above the agar, with a y-offset to the target worm (IP). The linear actuator vertically lowers the pick until contacting the agar surface (TP) as perceived by the capacitive touch sensor. Next, Servo 2 horizontally swipes the pick on the agar surface. Next, Servos 1 and 3 act simultaneously to perform a curved motion for picking up the target animal using the outer side of the wire loop [final position (FP)]. During phases i-iii, the motor speeds are maximized. To put down the animal, the pick is positioned above the agar surface (IP). The linear actuator lowers the pick until touching (TP), monitored by the capacitive touch sensor. Next, Servo 2 horizontally swipes the pick on the agar surface, during which the worm detaches from the wire; for some cases, the animal sticks to the inner side of the wire loop, and the moving stage swipes the pick to −x. Finally, Servo 1 raises the pick from the agar (FP). The motor speeds are tuned down in different phases, and waiting times are added to the phase transitions.

### Measurement of Viability After Automated and Manual Picking

The study manually measured animals' viability 24 hours after robotic and manual picking. The study classified the animals into three categories: alive, dead, and escaped. An animal was classified as alive if it moved actively; dead if it lost mobility; and escaped if one could not find it on the plate. For the paralyzed mutant unc-13 (CB1091), the study determined its viability by observing pharyngeal pumping.

### Measurement of Success Rates for the Pick-Up and Put-Down Procedures

To measure how effectively the system picks up and puts down animals, the study manually verified the success of individual pick-up and put-down attempts through the live low-magnification image stream. For accurate manual verifications, the study limited the number of animals to <30 per plate to reduce the probability of picking from and putting down in crowded areas. A pick-up attempt was deemed successful if the animal disappeared from the FOV; failed, if the animal remained in the FOV; and vice versa for verifying a put-down attempt. The success rates of the pick-up and put-down attempts were calculated by the number of successes divided by the number of attempts made.

WormPickerControl—For WormPicker to perform useful work, the basic elements of identifying and transferring animals need to be combined to form multistep genetic procedures. For this purpose, the study developed system control software, WormPickerControl. This software is written in Python for the frontend and C++ for the backend. The frontend is an API through which the user writes scripts to specify tasks to be performed by WormPicker. The study developed utilities in the API for initiating resources, generating scripts, managing a set of scripts, and sending the commands to the backend. The backend contains a library of source scripts, WormPickerLib, each controlling the hardware to carry out specific tasks. The user can access WormPickerLib through the API and has the flexibility to combine a set of scripts for generating custom protocols. To further improve the speed, the study set up multiple threads in the backend for separately handling image acquisition, image processing, hardware control, and script execution.

WormPickerControl contains a database, storing information in CSV format, through which the automated system catalogs a set of plates. The study constructed a Mask-RCNN segmentation server, written in Python, responsible for segmenting images acquired from different cameras. The server imports Mask-RCNN models from multiple locally saved PTH files. The study built client-server sockets connecting WormPickerLib to the segmentation server, through which images acquired by the hardware are sent to the corresponding Mask-RCNN models, and the inference results are sent back to the software for subsequent processing.

WormPickerLib is a library containing source scripts for performing various procedures, ranging from simple to complex. According to their complexity, elements in the library are categorized into low-, mid-, and high-level scripts. The low-level scripts enable the system to execute basic actions, such as sterilizing the pick, finding a worm with some desired phenotypes, picking, and putting down the animal. The mid-level scripts are composed of multiple low-level scripts chained in series, e.g. scripts to pick multiple animals with some desired phenotypes from a source to a destination. The high-level scripts are one iteration above the mid-level scripts, enabling the system to carry out complete C. elegans genetic procedures. The high-level scripts consist of a group of mid-level scripts arranged in a timed and conditional manner. The user has the flexibility to develop custom procedures using the elements in WormPickerLib.

The study included high-level scripts (CrossWorms, SingleWorms, and ScreenPlates) for instructing WormPicker to set up the mating for P0, single F1s, and screen F2s. The execution of CrossWorms relied on calling the mid-level script PickNWorms twice, each time for picking a certain number of males or hermaphrodites to a plate. Similarly, PickNWorms was executed in a sequential manner by iterating through a set of low-level scripts, which combined the basic actions of imaging and manipulation.

Strains—The study cultivated all the C. elegans strains used in this study at 20° C. on nematode growth medium plates with OP50 bacteria using the standard methods. All the experiments were carried out at room temperature.

### Configuration of Certain Implementations

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic. For example, circuit A communicably “coupled” to circuit B may signify that the circuit A communicates directly with circuit B (i.e., no intermediary) or communicates indirectly with circuit B (e.g., through one or more intermediaries).

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the form disclosed. Many modifications and variations will be apparent to those of ordinary skill in the art without departing from the scope and spirit of the present disclosure.

1. A system comprising:
an articulated robotic arm comprising at least one end effector including a plate manipulator;
a plurality of plates including a first plate having a first diameter, the first plate comprising a target organism, wherein a first lid corresponding to the first plate is removably coupled to the first plate, the first lid having a second diameter that is larger than the first diameter such that the first lid covers the first plate;
a first shelf array comprising a plurality of shelving units arranged within reach of the plate manipulator of the robotic arm, the first shelf array comprising:
a first plate shelf configured to retain the first plate, the first plate shelf defining a first plate holder opening having a third diameter, wherein the third diameter of the first plate holder opening is less than the first diameter of the first plate; and
a first lid shelf configured to retain the first lid, the first lid shelf defining a first lid holder opening having a fourth diameter, wherein the fourth diameter of the first lid holder opening is less than the second diameter of the first lid and greater than the first diameter of the first plate; and
a working platform for organizing and supporting the plurality of plates, the working platform comprising an upper surface defining a plurality of plate stations,
wherein the plate manipulator of the robotic arm is moveable to engage with the first plate to (i) remove the first plate from the first plate shelf, (ii) deposit the first lid from the first plate onto the first lid shelf, and (iii) move the first plate to a first plate station of the plurality of plate stations on the working platform without disengaging from the first plate.

2. The system of claim 1, wherein the plurality of plates further includes a second plate with a corresponding second lid, wherein the first shelf array further comprises a second plate shelf configured to retain the second plate and a second lid shelf configured to retain the second lid,
wherein the plate manipulator of the robotic arm is moveable to engage with the second plate to (i) remove the second plate from the second plate shelf, (ii) deposit the second lid from the second plate onto the second lid shelf, and (iii) move the second plate to a second plate station of the plurality of plate stations on the working platform without disengaging from the first plate.

3. The system of claim 1, wherein the plate manipulator includes a plate manipulator width that is less than the third diameter of the first plate holder opening such that the plate manipulator can pass through the first plate holder opening and the first lid holder opening without interference.

4. The system of claim 1, wherein, when removing the first plate from the first plate shelf, the plate manipulator moves upward through the first plate holder opening to engage with a base of the first plate.

5. The system of claim 4, wherein, when depositing the first lid onto the first lid shelf, the plate manipulator moves downward through the first lid holder opening such that the first lid shelf engages with and removes the first lid while the first plate passes through the first lid holder opening with the plate manipulator.

6. The system of claim 1, wherein the plurality of plate stations includes corresponding openings defined in the upper surface of the working platform, wherein a diameter of the openings of the plurality of plate stations are smaller than the first diameter of the first plate and larger than a plate manipulator width of the plate manipulator.

7. The system of claim 6, wherein, when moving the first plate to the first plate station, the plate manipulator moves through a first opening defined by the first plate station to deposit the first plate onto the first plate station.

8. The system of claim 1, wherein the at least one end effector further comprises a wire-loop picking tool comprising a wire loop electrically coupled to a power source to sterilize the wire-loop between uses, wherein each of the plate manipulator and the wire-loop picking tool is moveable between an active position and an inactive position relative to the robotic arm.

9. The system of claim 8, wherein the at least one end effector further comprises a camera adjacent to the wire-loop picking tool.

10. The system of claim 9, wherein the camera is configured to identify the target organism from the first plate and the wire-loop picking tool is configured to retrieve the target organism from the first plate based on information from the camera.

11. The system of claim 1, further comprising a controller in communication with the robotic arm, wherein the controller comprises a processor and a memory storing instructions thereon that, when executed by the processor, cause the robotic arm to perform a plate manipulation operation of an organism manipulation operation.

12. The system of claim 11, wherein the memory further stores a location for each of the plurality of plates held within the array of shelves.

13. The system of claim 1, further comprising:
an imaging system located under the working platform, the imaging system comprising at least one camera moveable along one or more rails, wherein the at least one camera is alignable with any one of the plurality of plate stations and a corresponding opening thereof to capture images of a plate therein.

14. The system of claim 13, wherein the imaging system further comprises a microscope coupled with a fluorescent imaging apparatus.

15. The system of claim 13, further comprising a controller in communication with the robotic arm and the imaging system, the controller comprising a processor and a memory storing instructions thereon that, when executed by the processor, cause the robotic arm to perform a plate manipulation operation of an organism manipulation operation,
wherein images captured by the imaging system are delivered to the controller and analyzed to identify the target organism or attributes thereof.

16-19. (canceled)

20. The system of claim 15, wherein the controller is configured to identify, via machine vision algorithms and artificial intelligence, one or more phenotypes of the target organism.

21-23. (canceled)

24. A method of categorizing a target organism, the method comprising:
(i) providing a system comprising:
a plurality of plates including a first plate containing a target organism and a first lid removably coupled to the first plate;
a first shelf array configured to house the plurality of plates, the first shelf array comprising a first plate shelf and a first lid shelf;
a working platform comprising a plurality of plate stations arranged along an upper surface and configured to hold the plurality of plates, the working platform defining a lower cavity accessible at least via an opening corresponding to each of the plurality of plate stations;
an imaging system movable along one or more rails disposed within the lower cavity, the imaging system comprising at least one optical device;
a robotic arm comprising at least one end effector moveable between the array of shelves and the working platform; and
a controller in communication with the robotic arm and the imaging system;
(ii) retrieving, via the at least one end effector of the robotic arm, the first plate from the first shelf;
(iii) depositing, via the at least one end effector of the robotic arm, the first plate onto a first plate station of the plurality of plate stations of the working platform;
(iv) actuating, via the controller, the imaging system to align the at least one optical device with the first plate;
(v) capturing, via the at least one optical device, images of the target organisms in the first plate; and
(vi) identifying, via the controller, an attribute of the target organism.

25. The method of claim 24, wherein depositing the first plate onto the first plate station comprises:
moving the at least one end effector to the first plate shelf housing the first plate, wherein the at least one end effector is a plate manipulator;
lifting, via the plate manipulator, the first plate and the first lid off of the first plate shelf;
moving the first plate through an opening of the first lid shelf to deposit the first lid on the first lid shelf, exposing the contents of the first plate; and
moving the first plate to the first plate station.

26. The method of claim 24, wherein identifying an attribute of the target organism comprises:
analyzing, via machine vision and/or artificial intelligence systems, images of the target organisms; and
identifying one or more phenotypes of the target organism.

27. The method of claim 24, wherein the at least one end effector further comprises a wire-loop picking tool, the method further comprising:
moving, via the robotic arm, the wire-loop picking tool into proximity with the first plate;
identifying, via the at least one optical device of the imaging system or a camera coupled to the wire-loop picking tool, a first target organism;
contacting the first target organism with the wire-loop picking tool; and
moving and depositing, via the robotic arm, the first target organism into a second plate on a second plate station.
